(19) United States
(12) Patent Application Publication
(10) Pub. No.: US 2026/0060561 A1
(43) Pub. Date: Mar. 5, 2026

(54) TECHNIQUES FOR UTILIZING THE VARIABILITY OF HEART RATE VARIABILITY

(71) Applicant: Oura Health Oy, Oulu (FI)

(72) Inventors: Anna Iashina, Tampere (FI); Mari Pauliina Karsikas, Oulu (FI); Emmi Maria Johanna Antikainen, Pirkkala (FI); Iman Alikhani, Espoo (FI)

(21) Appl. No.: 19/251,080

(22) Filed: Jun. 26, 2025

Related U.S. Application Data
(60) Provisional application No. 63/665,871, filed on Aug. 30, 2024.

(51) Int. Cl. A61B 5/024; A61B 5/00

(52) U.S. Cl. CPC A61B 5/02405; A61B 5/02438; A61B 5/4806; A61B 5/4884; A61B 5/7267; A61B 5/7435

(57) ABSTRACT

Methods, systems, and devices for utilizing the variability of heart rate variability (HRV) are described. A system may acquire heart rate variability (HRV) data measured from a user continuously via a wearable device throughout time intervals that the user is asleep. The system may identify the HRV data and determine the variability metric of the HRV data for the time intervals that the user is asleep. The system may then input the variability metric into a machine learning model that is trained to calculate physiological parameters of the user based on weighting the HRV data in accordance with one or more predictive weights that are based on the variability metric. The system may transmit an instruction for the user device to display the calculated physiological parameters and a recommendation for actions to be taken by the user to improve the variability metric of the HRV data.

### CROSS REFERENCE

The present application for patent claims priority to U.S. Provisional Patent Application No. 63/665,871 by IASHINA et al., entitled “TECHNIQUES FOR UTILIZING THE VARIABILITY OF HEART RATE VARIABILITY,” filed Aug. 30, 2024.

### FIELD OF TECHNOLOGY

The following relates to wearable devices and data processing, including techniques for utilizing the variability of heart rate variability (HRV).

### BACKGROUND

Some wearable devices may be configured to collect data from users associated with heart rate, motion data, temperature data, photoplethysmogram (PPG) data, and the like. In some cases, some wearable devices may perform various actions, such as providing certain health insights to users based on acquired physiological data in order to assist the user with improving their overall health. However, conventional techniques implemented by wearable devices are deficient.

Aspect(s) of the present disclosure are set out in the independent claim(s). Other aspects and features of the present disclosure are set out in the dependent claims and in the description below.

### BRIEF DESCRIPTION OF THE DRAWINGS

Implementations of the present disclosure are described below, by way of example, with reference to the accompanying drawings, in which:

FIG. 1 illustrates an example of a system that supports techniques for utilizing the variability of heart rate variability (HRV) in accordance with aspects of the present disclosure.

FIG. 2 illustrates an example of a system that supports techniques for utilizing the variability of HRV in accordance with aspects of the present disclosure.

FIG. 3 shows examples of timing diagrams that supports techniques for utilizing the variability of HRV in accordance with aspects of the present disclosure.

FIG. 4 shows an example of a timing diagram that supports techniques for utilizing the variability of HRV in accordance with aspects of the present disclosure.

FIG. 5 shows an example of a graphical user interface (GUI) that supports techniques for utilizing the variability of HRV in accordance with aspects of the present disclosure.

FIG. 6 shows a block diagram of an apparatus that supports techniques for utilizing the variability of HRV in accordance with aspects of the present disclosure.

FIG. 7 shows a block diagram of a wearable device manager that supports techniques for utilizing the variability of HRV in accordance with aspects of the present disclosure.

FIG. 8 shows a diagram of a system including a device that supports techniques for utilizing the variability of HRV in accordance with aspects of the present disclosure.

FIGS. 9 and 10 show flowcharts illustrating methods that support techniques for utilizing the variability of HRV in accordance with aspects of the present disclosure.

Like reference numerals are used for like components throughout the detailed description.

### DETAILED DESCRIPTION

The present disclosure will first be described in general terms before implementations of the disclosure are described by way of example with reference to the drawings. The word “may” is used to refer to a feature that is optional, i.e., in some implementations of the disclosure, the feature is present, and in some implementations of the disclosure, the feature is not present.

Some wearable devices may be configured to collect physiological data from users, such as temperature data, heart rate data, and the like. For example, a wearable device may collect heart rate measurements and corresponding heart rate variability (HRV) measurements from a user, where HRV is a measure of fluctuation (e.g., variability) of time intervals between adjacent heartbeats. HRV may be used to calculate various physiological parameters, such as the quality of the user's sleep, relative level of stress or relaxation of the user, and the like. It is generally understood that higher HRV indicates relatively better overall health (e.g., lower stress), and lower HRV indicates relatively poorer overall health (e.g., higher stress).

However, HRV metrics are typically calculated as an average of nighttime HRV readings, meaning HRV may be susceptible to outliers. As such, a calculated value of the user's HRV may not necessarily be indicative of the current state of the user (e.g., may not provide the most accurate insights into physiological changes and potential stressors affecting an individual's recovery and overall health). For example, due to outlier HRV values, a calculated high HRV value may not necessarily be a positive indication of the user's state, while a calculated low HRV value may not necessarily be a negative indication of the user's state.

Accordingly, aspects of the present disclosure are directed to systems and methods for calculating a variability metric for the HRV data by monitoring the variation of HRV data during the night. The variability metric for HRV data may indicate how much the user's HRV fluctuates and/or varies over time, where high variability metrics indicate larger swings in the user's HRV data, and low variability metrics indicate relatively constant (e.g., consistent) HRV data. As such, the variability metric of HRV data may be used to determine how reliable or representative the user's calculated HRV value is of their overall health.

As described herein, a wearable device may be used to acquire physiological data from a user, such as throughout the day and overnight as the user sleeps. A user device, the wearable device, or both, may identify a set of HRV values associated with a set of time intervals of the sleep period (e.g., during the night) based on the HRV data collected via the wearable device throughout the sleep period. Further, the wearable device, the user device, or both, may calculate some “variability metric” that is associated with the relative level of variability of the set of HRV values during the sleep period. That is, some aspects of the present disclosure focus on determining the variability metric associated with the set of HRV values of the sleep period. In such cases, the variability metric is associated with one or more changes in the set of HRV values throughout the sleep period.

The variability metric for the HRV data may be calculated by using one or more calculation metrics including, but not limited to, an interquartile range (IQR) of HRV, a coefficient of variation (CV), a standard deviation, and the like. The calculated variability metric may then be used to weight the user's HRV data when calculating various physiological scores or physiological parameters for the user. For instance, when calculating the user's Readiness Score, HRV data may be weighted less when the variability metric is high (e.g., indicating that the HRV data may be relatively unreliable), whereas HRV data may be weighted more heavily when the variability metric is low (e.g., indicating relatively constant HRV data).

The variability metric may be inputted into a machine learning model where the machine learning model is trained to calculate one or more physiological parameters of the user based on weighting the set of HRV values in accordance with one or more predictive weights. The predictive weights may be based on the variability metric. In other words, the user's HRV data may be weighted more or less based on the variability metric of the HRV data, as the variability metric may be a relative measure of how “trustworthy” the user's HRV data is for evaluating other physiological parameters. In such cases, the variability metric may be used as an input into other models such as chronic stress, Readiness Score, sleep staging, and the like. For instance, high variability metrics may be indicative of stress or overtraining, and may therefore be used for calculating a user's cumulative stress over time. As described herein, a user may alter one or more behaviors in accordance with the cumulative stress level, such as to reduce the risk of burn-out, chronic illness, etc., which may help improve their overall health based on the current mental, physical, and emotional state of the user.

The system may transmit, using the one or more processors, one or more signals to the user device, where the one or more signals include an instruction for a graphical user interface (GUI) of the user device to display the one or more calculated physiological parameters and a recommendation for one or more actions to be taken by the user to improve the variability metric of the HRV data. A user may alter one or more behaviors in accordance with the calculated physiological parameters and the recommendation, such as to improve the variability metric of HRV, which may help improve their overall health based on the current mental, physical, and emotional state of the user. For example, the user device may provide the user instructions regarding how the user may improve the variability metric or positive feedback instructing the user to maintain a current variability metric. A user may alter one or more behaviors in accordance with the instructions, which may help improve their ability to endure stress, for example, and improve their overall health. Additionally, or alternatively, the system may automatically adjust the user's surroundings (e.g., adjust a smart thermostat, turn off a TV while the user is sleeping, etc.) based on the determined variability metric. That is, the system may automatically make adjustments to the user's surroundings based on the determined variability metric in order to help improve the user's overall physiological health (e.g., to improve the variability metric of the user in the future).

Aspects of the disclosure are initially described in the context of systems supporting physiological data collection from users via wearable devices. Additional aspects of the disclosure are described in the context of example timing diagrams and an example GUI. Aspects of the disclosure are further illustrated by and described with reference to apparatus diagrams, system diagrams, and flowcharts that relate to techniques for utilizing the variability of HRV.

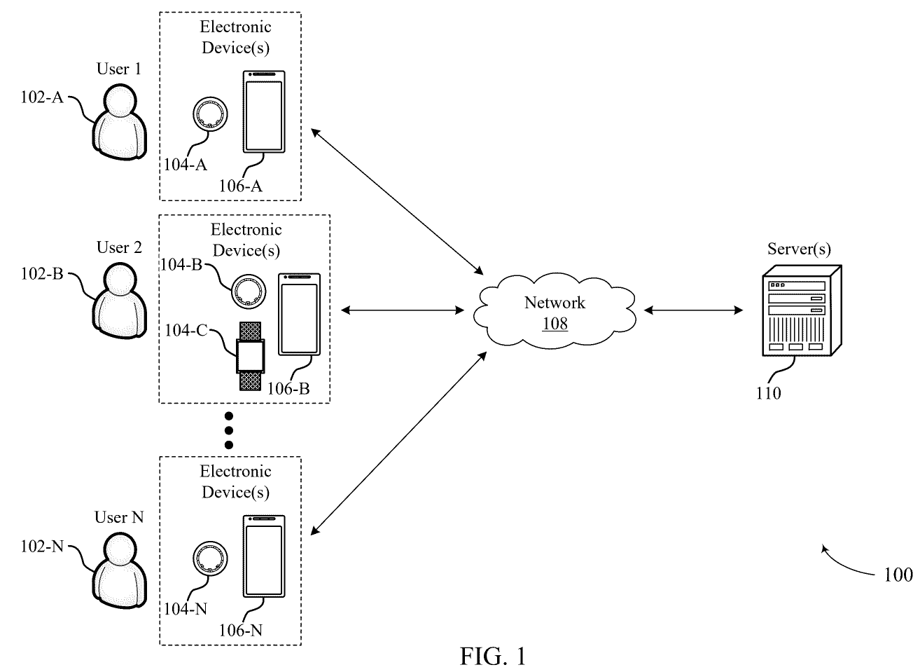

FIG. 1 illustrates an example of a system 100 that supports techniques for utilizing the variability of HRV in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices, such as those known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user's 102 finger, a wrist computing device (e.g., a smart watch, fitness band, or bracelet) configured to be worn on a user's 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing, or may otherwise be maintained within the vicinity of the user 102. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, HRV, actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), mobile device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols. Moreover, in some cases, the wearable device 104 and the user device 106 may be included within (or make up) the same device. For example, in some cases, the wearable device 104 may be configured to execute an application associated with the wearable device 104, and may be configured to display data via a GUI.

In some implementations, the rings 104 (e.g., wearable devices 104) of the system 100 may be configured to collect physiological data from the respective users 102 based on arterial blood flow within the user's finger. In particular, a ring 104 may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 100 may be configured to collect physiological data from the respective users 102 based on blood flow diffused into a microvascular bed of skin with capillaries and arterioles. For example, the system 100 may collect PPG data based on a measured amount of blood diffused into the microvascular system of capillaries and arterioles. In some implementations, the ring 104 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the ring 104 has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a ring 104 has been found to exhibit superior performance as compared to wearable devices worn on the wrist, as the ring 104 may have greater access to arteries (as compared to capillaries), thereby resulting in stronger signals and more valuable physiological data.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the system 100 may detect periods of time that a user 102 is asleep, and classify periods of time that the user 102 is asleep into one or more sleep stages (e.g., sleep stage classification). For example, as shown in FIG. 1, User 102-a may be associated with a wearable device 104-a (e.g., ring 104-a) and a user device 106-a. In this example, the ring 104-a may collect physiological data associated with the user 102-a, including temperature, heart rate, HRV, respiratory rate, and the like. In some aspects, data collected by the ring 104-a may be input to a machine learning classifier, where the machine learning classifier is configured to determine periods of time that the user 102-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 102-a via a GUI of the user device 106-a. Sleep stage classification may be used to provide feedback to a user 102-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 100 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 102-a via the wearable device 104-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 102 to generate tailored, individualized circadian rhythm adjustment models that are specific to each respective user 102.

In some aspects, the system 100 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the system 100 may support techniques for calculating a variability metric for HRV. For example, the system 100 may support techniques for utilizing the variability metric for HRV for a user. In particular, techniques described herein support a wearable device 104, such as a wearable ring device 104 as described with reference to FIG. 1. For example, a wearable device 104 may include an inner housing 105 configured to house a sensor module that includes one or more sensors that are configured to acquire physiological data from a user 102. The one or more sensors of the wearable device 104 may obtain physiological measurements from the user (e.g., temperature sensors, additional LED-PD sensors used for measuring heart rate, oxygen saturation, one or more sensors that a device may use to detect whether a user is asleep, active, or the like).

For example, the one or more sensors of the wearable device 104 may acquire physiological data from a user throughout one or more time intervals, where the physiological data may include heart rate data, HRV data, motion data, or a combination thereof. In some examples, the time intervals include an asleep interval (e.g., sleep period) during which the user is asleep. The user device 106 may receive the physiological data (e.g., including the physiological data measured during the one or more time intervals) from the wearable device 104.

In some cases, the one or more sensors of the wearable device 104 are configured to acquire the physiological data from the user based on arterial blood flow, body temperature, etc. In some implementations, the one or more sensors of the wearable device 104 are configured to acquire the physiological data (e.g., including PPG data) from the user based on blood flow that is diffused into the microvascular bed of skin with capillaries and arterioles. The one or more sensors of the wearable device 104 may be an example of photodetectors from the PPG system, temperature sensors, motion sensors, galvanic sensors, and other sensors.

While much of the present disclosure describes one or more components in the context of a wearable ring device, aspects of the present disclosure may additionally, or alternatively, be implemented in the context of other wearable devices. For example, in some implementations, the one or more components described herein may be implemented in the context of other wearable devices, such as bracelets, watches, necklaces, piercings, and the like. For example, the wearable device 104 may surround a finger, wrist, ankle, earlobe, or the like of a user.

For example, as noted previously herein, the wearable device 104 of the system 100 may be worn by a user to collect data from the user, including temperature data, sleep data, recovery data, activity data, PPG data, heart rate data, HRV data, respiratory rate data, blood pressure data, blood glucose data, and the like. The wearable device 104 of the system 100 may collect the physiological data from the user based on sensors and measurements extracted from arterial blood flow (e.g., using PPG signals). In some cases, the wearable device 104 may collect the physiological data from the user based on measurements extracted from capillary blood flow, arteriole blood flow, or both. The physiological data may be collected continuously.

In some implementations, the one or more sensors of the wearable device 104 may sample the user's PPG data continuously throughout the day and night. Sampling at a sufficient rate (e.g., one sample per minute) throughout the day and/or night may provide sufficient PPG data for analysis described herein. In some implementations, the wearable device 104 may continuously acquire PPG data (e.g., at a sampling rate). In some examples, even though PPG data is collected continuously, the system 100 may leverage other information about the user that it has collected or otherwise derived (sleep stage, activity levels, illness onset, stress, etc.) to select a representative PPG for a particular day that is an accurate representation of the underlying physiological phenomenon.

In some aspects, physiological data collected via the wearable devices 104 (e.g., HRV data via the PPG data) may be used to determine a variability metric associated with the set of HRV values of the sleep period. The variability metric may be associated with one or more changes in the set of HRV values throughout the sleep period. In some cases, the variability metric may be inputted into a machine learning model. The machine learning model may be trained to calculate one or more physiological parameters of the user based on weighting the set of HRV values in accordance with one or more predictive weights that are based on the variability metric. The system 100 may transmit one or more signals to the user device that include an instruction for the GUI of the user device to display the one or more calculated physiological parameters and a recommendation for one or more actions to be taken by the user to improve the variability metric of the HRV data.

It should be appreciated by a person skilled in the art that one or more aspects of the disclosure may be implemented in a system 100 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

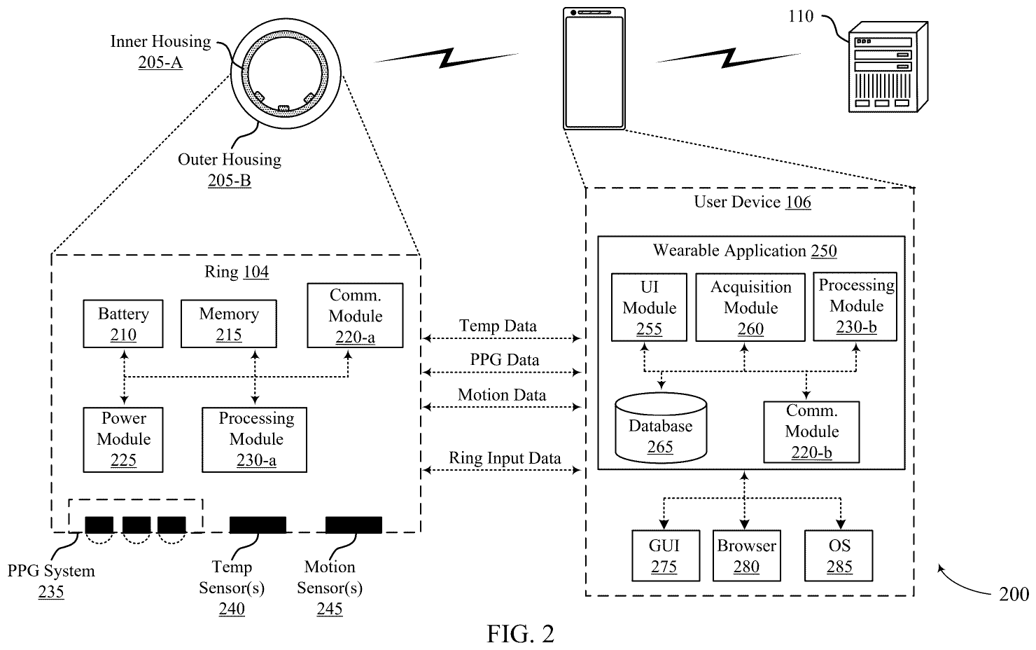

FIG. 2 illustrates an example of a system 200 that supports techniques for utilizing the variability of HRV in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205 that may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user's finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-b. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user's finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or that supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during charging, and under voltage during discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user's finger (e.g., skin temperature). In some implementations, the temperature sensor 240 may contact the user's skin. In other implementations, a portion of the housing 205 (e.g., the inner housing 205-a) may form a barrier (e.g., a thin, thermally conductive barrier) between the temperature sensor 240 and the user's skin. In some implementations, portions of the ring 104 configured to contact the user's finger may have thermally conductive portions and thermally insulative portions. The thermally conductive portions may conduct heat from the user's finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user's temperature over time. For example, the processing module 230-a may sample the user's temperature according to a sampling rate. An example sampling rate may include one sample per second, although the processing module 230-a may be configured to sample the temperature signal at other sampling rates that are higher or lower than one sample per second. In some implementations, the processing module 230-a may sample the user's temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user's finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user's finger (e.g., any finger). For example, one or more temperature sensors 240 on the ring 104 may acquire a user's temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 235 may indicate the perfusion of blood in the illuminated region. For example, the PPG signal may indicate blood volume changes in the illuminated region caused by a user's pulse pressure. The processing module 230-a may sample the PPG signal and determine a user's pulse waveform based on the PPG signal. The processing module 230-a may determine a variety of physiological parameters based on the user's pulse waveform, such as a user's respiratory rate, heart rate, HRV, oxygen saturation, and other circulatory parameters.

In some implementations, the PPG system 235 may be configured as a reflective PPG system 235 where the optical receiver(s) receive transmitted light that is reflected through the region of the user's finger. In some implementations, the PPG system 235 may be configured as a transmissive PPG system 235 where the optical transmitter(s) and optical receiver(s) are arranged opposite to one another, such that light is transmitted directly through a portion of the user's finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user's heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBIs. The processing module 230-a may store the HRV values over time in the memory 215. Moreover, the processing module 230-a may determine the user's respiratory rate over time. For example, the processing module 230-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user's finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user's physiological parameters may be measured by sensors included on a ring 104, other devices may measure a user's physiological parameters. For example, although a user's temperature may be measured by a temperature sensor 240 included in a ring 104, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

In some cases, the wearable device 104 and the user device 106 may be included within (or make up) the same device. For example, in some cases, the wearable device 104 may be configured to execute the wearable application 250, and may be configured to display data via the GUI 275.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, data collected by the wearable device 204, and/or analyses performed by the wearable device 204, the user device 206, and/or the servers 210 (such as “variability metrics” of HRV data), may be used to adjust operational parameters of the wearable device 204. For example, based on a determined heart rate of the user and/or a determined activity state of the user, the wearable device 204 may adjust a sampling rate for measuring the user's heart rate, and/or may activate or deactivate certain sensors and/or physiological measurements (e.g., deactivate SpO2 measurements when the user is engaged in physical activity, or otherwise exhibits an activity/movement level above some threshold). By way of another example, the user device 206 and/or the servers 210 may calculate a Readiness Score for the user, and may deactivate or disable activity measurements performed by the wearable device 204 in cases where the Readiness Score is below some threshold (in order to reduce power consumption and conserve battery at the wearable device 204, and/or to disincentivize the user from performing rigorous activity when their Readiness Score is below the threshold value). In this regard, any measurements, calculations, and/or analyses performed by the various devices within the system 200 (e.g., wearable device 204, user device 206, servers 210) may be used by the system 200 to control and/or adjust the operational parameters of the wearable device 204.

Operational parameters that may be controlled/adjusted at the wearable device 204 based on collected data and/or analyses performed by the system 200 may include, but are not limited to, a periodicity/frequency that measurements are performed (e.g., sampling rate), a power level or intensity of LEDs, algorithms used to analyze data at the wearable device 204, what types of measurements are performed (e.g., enabling/disabling specific sensors or types of measurements), a periodicity or frequency that the wearable device 204 transmits data to the user device 206, or any combination thereof. Adjusting operational parameters of the wearable device 204 based on collected data and/or analyses performed by the system 200 may reduce power consumption and improve battery performance at the wearable device 204, and may lead to higher quality data collected by the wearable device 204, thereby enabling the system 200 to perform more accurate and reliable analyses/diagnoses of the user's physiological parameters, and leading to better guidance and insights that may enable the user to improve their overall health.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 200 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, physiological data collected via the wearable devices 104 (e.g., HRV data) of the system 200 may be used to calculate a variability metric of the HRV data, where the variability metric of the HRV data is indicative of one or more changes in the HRV data. For example, high variability metrics indicate larger swings in the user's HRV data, and low variability metrics indicate relatively constant (e.g., consistent) HRV data. As such, the variability metric of HRV data may be used to determine how reliable or representative the user's calculated HRV value is of their overall health. The user device may provide the user instructions regarding how the user may improve the variability metric, or positive feedback instructing the user to maintain a current variability metric such that the user may alter one or more behaviors in accordance with the instructions, which may help improve their overall health.

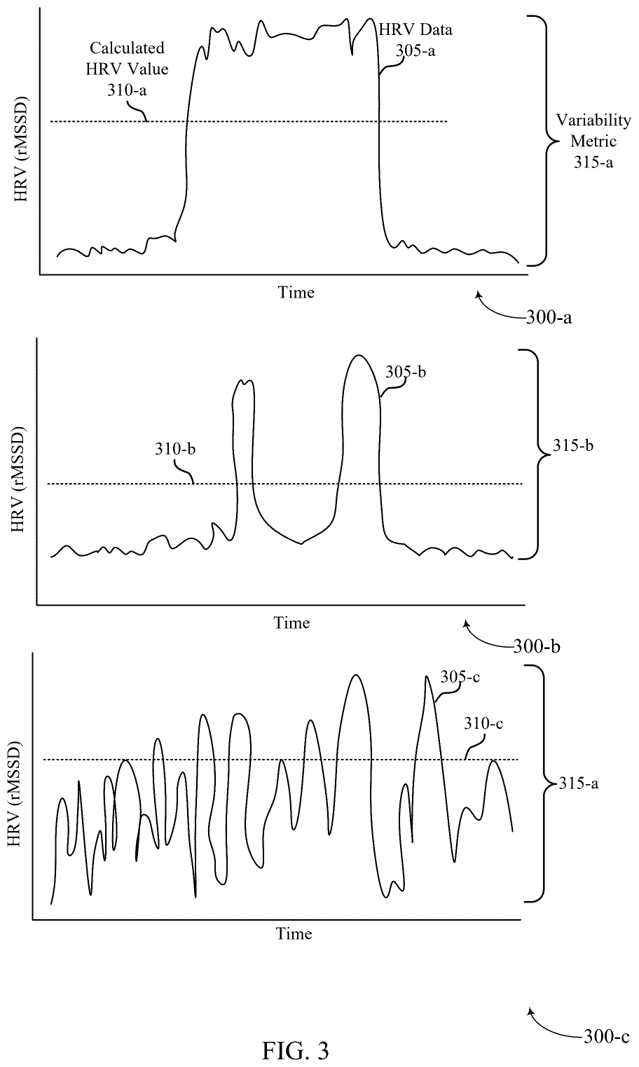

FIG. 3 shows examples of timing diagrams 300 that supports techniques for utilizing the variability of HRV in accordance with aspects of the present disclosure. The timing diagrams 300 may implement, or be implemented by, aspects of the system 100, system 200, or both. For example, in some implementations, the timing diagrams 300 may be displayed to a user via the GUI 275 of the user device 106, as shown in FIG. 2.

The timing diagrams 300 illustrate a how the user's HRV measurements (e.g., HRV data 305) change over time. In this regard, the curved lines illustrated in the timing diagrams 300 may be understood to refer to the “HRV data 305.”

As noted previously herein, a user's “HRV value” is typically calculated as some sort of average value of the user's HRV value overnight (as they are asleep). In this regard, the dashed horizontal lines illustrated in the timing diagrams 300 may illustrate “average” or “calculated” HRV values (e.g., calculated HRV value 310) based on the respective HRV data 305.

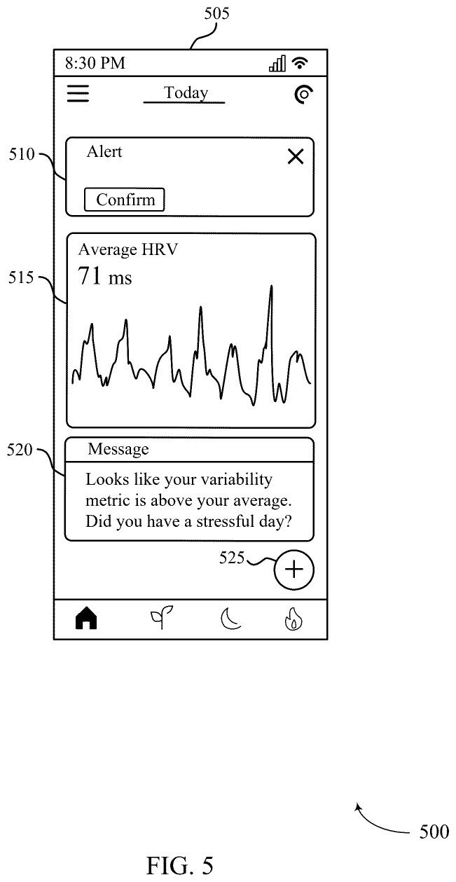

The calculated HRV value 310 is typically calculated as an average of nighttime HRV readings (e.g., the HRV data 305), meaning the calculated HRV value 310 may be susceptible to outliers. The calculated HRV value 310 may be presented to the user via the GUI of the user device, as described with reference to FIG. 5. However, the singular value of the calculated HRV value 310 may not necessarily be indicative of the current state of the user (e.g., may not provide the most accurate insights into physiological changes and potential stressors affecting an individual's recovery and overall health). For example, due to outlier HRV values from the HRV data 305, a calculated high HRV value 310 may not necessarily be a positive indication of the user's state, while a calculated low HRV value 310 may not necessarily be a negative indication of the user's state. As such, the system may calculate a variability metric 315 that may be used to determine how reliable or representative the user's calculated HRV value 310 is of their overall health.

As described in further detail herein, the system may be configured to determine the variability metric 315 associated with the plurality of HRV values (e.g., HRV data 305) of the sleep period. The variability metric 315 is associated with one or more changes in the HRV data 305 throughout the sleep period. In such cases, the user's HRV data 305 throughout the night may be used to determine the variability metric 315.

A user's HRV data 305 may be a valuable metric used to assess the autonomic nervous system's activity by measuring the variation in the time intervals between consecutive heartbeats. Monitoring HRV data 305 during sleep provides insights into physiological changes and potential stressors affecting an user's recovery and overall health. Within-night variability of HRV (e.g., variability metric 315) refers to fluctuations in HRV metrics throughout a single night's sleep. The system may measure HRV data 305 with rMSSD of consecutive IBI and use average nighttime HRV (e.g., calculated HRV value 310) as the HRV balance contributor to the Readiness Score, for example, thereby making the variability metric 315 susceptible to outliers in within-night HRV data 305.

The system (e.g., ring 104, user device 106, server 110) may receive physiological data associated with a user from the wearable device. The physiological data may include at least HRV data 305. The HRV data 305 may be continuously collected by the wearable device. The physiological measurements may be taken continuously throughout the day and/or night. For example, in some implementations, the ring may be configured to acquire physiological data (e.g., temperature data, sleep data, heart rate, HRV data 305, respiratory rate data, MET data, and the like) continuously in accordance with one or more measurement periodicities throughout the entirety of each day/sleep day. In other words, the ring may continuously acquire physiological data from the user without regard to “trigger conditions” for performing such measurements. In some cases, continuous HRV measurement may capture HRV fluctuations (e.g., small or large fluctuations). For example, continuous HRV measurement may capture minute-to-minute or hour-to-hour HRV fluctuations that provide additional insight that may not be provided by other HRV measurements.

In some cases, the physiological data measured continuously may include at least PPG data (e.g., volumetric blood flow change). The PPG data may be continuously collected by the wearable device. The physiological measurements may be taken continuously throughout the day and/or night. For example, in some implementations, the ring may be configured to acquire physiological data (e.g., PPG data, HRV data 305) continuously in accordance with one or more measurement periodicities throughout the entirety of each day/sleep day. One or more pulses may be derived from the PPG signal (e.g., PPG data), and the HRV data 305 may be derived from the PPG pulse foot-to-pulse foot intervals.

In some implementations, the system may identify the HRV data 305 by observing a user's relative HRV data 305 for many days. In some cases, the HRV data 305 may be an example of nighttime HRV, daytime HRV, or both. With reference to timing diagrams 300, the calculated HRV value 310 may be an example of the average nighttime HRV that is continuously measured during a single sleep period (e.g., a previous night's sleep of the user). The sleep period may include the time interval that the user is asleep. In some cases, the calculated HRV value 310 may be an example of average nighttime HRV, average daytime HRV, or both.

The variability metric 315 may be calculated based on at least the HRV data 305 collected while the user is asleep. As described herein, the variability metric 315 may be calculated based on a variety of calculation techniques. For example, the variability metric 315 may be calculated using a interquartile range (IQR) of the HRV data 305. In such cases, the variability metric 315 may be calculated by determining the difference between the 75th percentile of HRV values (e.g., HRV data 305) within the night and the 25th percentile of HRV values (e.g., HRV data 305) within the night. For example, the system may determine a difference between a first percentile (e.g., 75th percentile) of the plurality of HRV values and a second percentile (e.g., 25th percentile) of the plurality of HRV values in response to identifying the plurality of HRV values.

Using the IQR of HRV to calculate the variability metric 315 may provide a measure of the spread or dispersion of HRV values (e.g., HRV data 305), thereby indicating the variability within the night's data. The IQR of HRV calculation technique may account for outliers of the HRV data 305 and provide a more robust representation of HRV variability (e.g., the variability metric 315).

In some cases, a higher IQR value may suggest increased physiological stress or disturbances during sleep. In such cases, the IQR of HRV calculation technique may be used to capture the nights following a physiologically stressful day as the calculation technique accounts for both high variability and low averages. That is, IQR of HRV and peaks in IQR of HRV may be better predictors of high cumulative stress and assessed risk of burnout when compared to other variability metric 315 calculation techniques, such as coefficient of variation, as described herein. The IQR of HRV normalized by average nighttime HRV may be used to make the night of a single user comparable to other users. In such cases, the normalized IQR may be beneficial for comparing data across different nights and/or different users. The IQR of HRV normalized may be an example of IQR (overnight HRV)/AVG (overnight HRV).

In some examples, the variability metric 315 may be calculated using a coefficient of variation. The coefficient of variation may be an example of the standard deviation of the HRV data 305 divided by the mean HRV data 305 (e.g., calculated HRV value 310), expressed as percentage. In such cases, the system may determine a coefficient of variation for the plurality of HRV values based on determining the standard deviation. The coefficient of variation may reflect the relative variability of HRV data 305 (e.g., the variability metric 315) within a night, irrespective of the scale of HRV data 305. The coefficient of variation calculation technique may be useful for comparing the variability metric 315 across different individuals or nights. In some cases, the coefficient of variation calculation technique may be better at capturing the nights with outliers in HRV data 305 which may be caused by arrhythmia or ectopic beats, and potentially more serious conditions.

In some examples, the variability metric 315 may be calculated using the standard deviation of the HRV data 305. In such cases, the system may determine a standard deviation of the plurality of HRV values in response to identifying the plurality of HRV values. The standard deviation calculation technique may measure the dispersion of HRV values (e.g., HRV data 305) around the mean within a night (e.g., the sleep period). The standard deviation may indicate the average distance of HRV values from the mean, thereby providing insight into the overall variability metric 315. The higher standard deviation values may suggest greater fluctuations in HRV data 305 throughout the night.

The variability metric 315 may be calculated using a quartile coefficient of dispersion. The quartile coefficient of dispersion calculation technique may be an example of a more robust calculation technique to outliers of the variability metric 315 as compared to other calculation techniques such as the coefficient of variation. The quartile coefficient of dispersion calculation technique may be useful for comparing variability metrics 315 across different users at night.

In some cases, the variability metric 315 may be calculated using a frequency domain analysis. The frequency domain analysis may utilize spectral analysis to assess the distribution of HRV power within frequency bands (e.g., high frequency bands and/or low frequency bands). The variability in power distribution across frequency bands may indicate changes in autonomic modulation during sleep stages of the user's sleep period.

In other examples, the variability metric 315 may be calculated using time domain parameters. The time domain parameters may include metrics such as SDNN (standard deviation of normal-to-normal intervals) and pNN50 (percentage of successive RR intervals differing by more than 50 ms). The time domain parameters may reflect an overall variability metric 315 and vagal tone modulation during a sleep period. By using at least one of the various calculation techniques for determining the variability metric 315, a comprehensive characterization of HRV data 305 dynamics within a single night may be achieved.

Although the system may be implemented by a ring and a user device, any combination of computing devices described herein may implement the features attributed to the system. In some cases, the system may smooth the HRV data 305. The missing values of HRV data 305 may be imputed (e.g., using a forecaster model, such as the Impute method from the Python package or a similar coding language library).

With reference to the timing diagrams 300, each timing diagram may illustrate the relationship between a user's HRV data 305, the calculated HRV value 310, and the variability metric 315. For example, timing diagram 300-a may be representative of a first user's HRV data 305-a, calculated HRV value 310-a, and the variability metric 315-a, timing diagram 300-b may be representative of a second user's HRV data 305-b, calculated HRV value 310-b, and the variability metric 315-b, and timing diagram 300-c may be representative of a third user's HRV data 305-c, calculated HRV value 310-c, and the variability metric 315-c. In such cases, each timing diagram 300 may include a similar average HRV value (e.g., calculated HRV value 310) but the variability metric 315 for each set of HRV data 305 may be different.

For example, the timing diagram 300-a may include a high calculated HRV value 310-a (e.g., average HRV value). As is evident from the HRV data 305-a, a user's HRV data 305-a may trend upward throughout a beginning portion of the sleep period, remain high throughout a portion of the sleep period, and then trend downward throughout an end portion of the sleep period. In such cases, the first user may have a relatively high variability metric 315-a due to the phases (e.g., portions) of HRV data 305-a with low HRV data 305-a at the beginning and end portions of the sleep period and high HRV data 305-a during a middle portion of the sleep period. For example, using the IQR calculation technique, the first user may have a relatively high variability metric 315-a, and using the coefficient of variation technique, the first user may have a relatively medium variability metric 315-a.

The timing diagram 300-b may include a slightly higher calculated HRV value 310-b (e.g., average HRV value) as compared to timing diagram 300-a. As is evident from the HRV data 305-b, a user's HRV data 305-b may include at least two peaks of HRV data 305-b during the sleep period but otherwise remain relatively consistent and lower during the sleep period. In such cases, the second user may have a relatively lower variability metric 315-b as compared to the variability metric 315-a. For example, using the IQR calculation technique, the second user may have a relatively low variability metric 315-b, and using the coefficient of variation technique, the second user may have a relatively high variability metric 315-b.

The timing diagram 300-c may include a high calculated HRV value 310-c (e.g., average HRV value) due to the variability of the HRV data 305-c during the sleep period. As is evident from the HRV data 305-c, a user's HRV data 305-c may include multiple peaks of HRV data 305-c during the sleep period without any HRV data 305-c that is maintained at a constant, consistent value during the sleep period. In such cases, the third user may have a relatively higher variability metric 315-c as compared to the variability metric 315-a and the variability metric 315-b. For example, using the IQR calculation technique, the third user may have a high variability metric 315-c, and using the coefficient of variation technique, the second user may have a high variability metric 315-c.

As described herein, the calculated high HRV value 310 from the HRV data 305 may not necessarily be a positive indication of the user's state because the high variability metric 315 may indicate larger swings in the user's HRV data 305. The HRV data 305 may be weighted less when the variability metric 315 is high (e.g., indicating that the HRV data 305 may be relatively unreliable). The variability metric 315 may provide valuable insights into autonomic nervous system regulation, stress responses, pregnancy complications, Readiness contributors (e.g., recovery index, HRV balance), and overall health status, as described herein, thereby facilitating personalized monitoring and intervention strategies.

In some implementations, the system may utilize the variability metric 315 to adjust operational parameters of the wearable device 104. That is, the calculated variability metric 3145 may be used to adjust parameters of the wearable device 104 itself (e.g., adjust how/when the wearable device 104 collects future physiological data). For example, the wearable device 104 may adjust an LED intensity or current applied to the LEDs based on the variability metric 315, and/or may adjust a sampling rate or frequency of the LEDs/PDs based on the variability metric 315. In particular, the system may utilize the variability metric 315 to improve an accuracy or reliability of future physiological data (e.g., future HRV data) collected by the wearable device 104. Similarly, in some cases, the system may trigger the wearable device 104 to perform certain types of measurements based on the determined variability metric 315.

Additionally, or alternatively, the system may automatically adjust the user's surroundings (e.g., adjust a smart thermostat, turn off a TV while the user is sleeping, etc.) based on the determined variability metric 315. That is, the system may automatically make adjustments to the user's surroundings based on the determined variability metric in order to help improve the user's overall physiological health (e.g., to improve the variability metric of the user in the future). In some cases, the system may continue to monitor the user's physiological data (e.g., HRV data 305) after making such changes to the user's surroundings to determine an effect of such changes, and to identify specific changes to the user's environment that positively (or negatively) affected the user's overall health (e.g., identify how changes to the user's environment affect the user's HRV data 305 and/or variability metric 315 going forward).

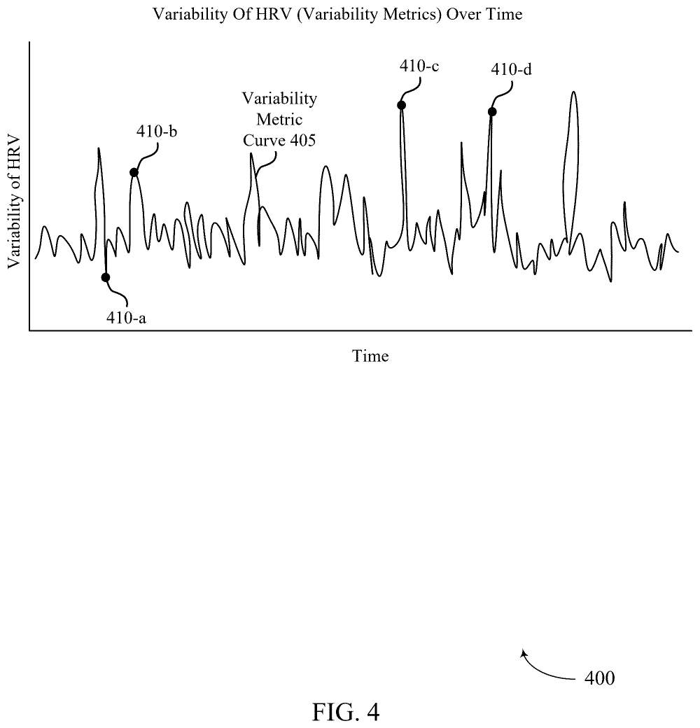

FIG. 4 shows an example of a timing diagram 400 that supports techniques for utilizing the variability of HRV in accordance with aspects of the present disclosure. The timing diagram 400 may implement, or be implemented by, aspects of the system 100, system 200, timing diagrams 300, or a combination thereof. For example, in some implementations, the timing diagram 400 may be displayed to a user via the GUI 275 of the user device 106, as shown in FIG. 2.

As described in further detail herein, the system may be configured to determine a user's variability metric over time, as represented by the variability metric curve 405. As noted previously herein, the respective variability metrics plotted via the a variability metric curve 405 may be associated with the plurality of HRV values of sleep periods of the user, where the respective variability metric values of the variability metric curve 405 are associated with one or more changes in the plurality of HRV values throughout the respective sleep periods. In some cases, the user's HRV data throughout the night may be used to determine the variability metric 405. By way of example, the variability metric curve 405 may include one variability metric per day over the course of multiple weeks.

As such, the variability metric curve 405 in the timing diagram 400 illustrates multiple variability metrics of the user over the course of some period of time (e.g., over a plurality of days, months, etc.). The timing diagram 400 may illustrate the variability metric curve 405 that illustrates variability metrics calculated for a user from sets of HRV values that were measured continuously throughout multiple sleep periods (e.g., each night's sleep for the plurality of days), as described with reference to FIG. 3. The variability metrics of the variability metric curve 405 may be calculated using the normalized IQR of HRV, as described with reference to FIG. 3.

The timing diagram 400 may include a first event 410-a, a second event 410-b, a third event 410-c, and a fourth event 410-d. The first event 410-a may be indicative of a start of vacation, and the second event 410-b may be indicative of an end of vacation. The third event 410-c may be indicative of a work presentation. The fourth event 410-d may be indicative of a late night social gathering. As described in further detail herein, by evaluating a user's variability metric(s) over time, the system may be able to more accurately and efficiently identify stressful events and other physiological conditions for the user, such as chronic stress, burnout, etc.

In some cases, the system may identify non-stressful events (e.g., first event 410-a and second event 410-b) when the user's HRV variability metric (as represented by the variability metric curve 405) is at or below a threshold value. For example, the system may identify a non-stress-inducing event experienced by the user during a time interval preceding the sleep period based on the variability metric curve 405 failing to exceed the threshold value. As described with reference to FIG. 4, the first event 410-a may be indicative of the start of a vacation where the user may be relaxed to start the vacation, as shown by a lower value of the variability metric curve 405 for that day. At the end of vacation, as shown via the second event 410-b, the value of the variability metric curve 405 may be higher than the value of the variability metric curve 405 at the first event 410-a, but the variability metric 405 may still be lower than the threshold value. In some cases, the threshold value may be determined based on a population distribution according to an age cohort (e.g., group). For example, the threshold value may be an example of a value for a 75th percentile of an age group spanning 20 to 30 years of age for a 22 year old user. As described previously herein, in some cases, the system may adjust operational parameters of the wearable device 104 and/or trigger adjustments to the user's surroundings (e.g., adjust a smart thermostat, turn on/off a TV, etc.) based on the variability metric satisfying (or failing to satisfy) some threshold value(s).

In such cases, the user device may display an indication of the non-stress-inducing event to the user. As described herein with reference to FIG. 5 the system may transmit one or more additional signals to the user device that cause the user device to display a prompt for the user to confirm the non-stress-inducing event. For example, the user device may display a prompt indicating “Looks like your variability metric is below your average. Did you have a relaxing day?” The system may receive, via the user device, a confirmation of the non-stress-inducing event. The machine learning model may be retrained to identify non-stress-inducing events for the user based on the HRV data measured from the user and based on inputting the non-stress-inducing events and the confirmation into the machine learning model.

In some cases, the system may identify stressful events (e.g., overtraining, stressful work days, and the like) when the value of the variability metric curve 405 spikes. The spike may be indicative of a maximum of the variability metric 405. For example, the system may identify a stress-inducing event experienced by the user during a time interval preceding the sleep period based on the variability metric 405 exceeding a threshold value. The third event 410-c may be indicative of a stressful event. In some cases, the fourth event 410-d may be indicative of a stressful event (e.g., an event that causes stress on the body). The variability metrics 405 for the third event 410-c and the fourth event 410-d may be above the threshold, where the third event 410-c and the fourth event 410-d may be examples of a stressful work day, overtraining for an activity, an activity that may induce stress on the user's physiological functions (e.g., a night of drinking alcoholic beverages, staying up late at a party, etc.), and the like.

In such cases, the user device may display an indication of the stress-inducing event to the user. As described further with reference to FIG. 5, the system may transmit one or more additional signals to the user device that cause the user device to display a prompt for the user to confirm the stress-inducing event. For example, the user device may display a prompt indicating “Looks like your variability metric is above your average. Did you have a stressful day?” The system may receive, via the user device, a confirmation of the stress-inducing event, and the machine learning model may be retrained to identify stress-inducing events for the user based on the HRV data measured from the user and based on inputting the stress-inducing event and the confirmation into the machine learning model.

In some cases, repeated spikes (e.g., maximums) of the variability metric curve 405 or a consistent elevated variability metric curve 405 above the threshold may be indicative of underlying long-term physical and or mental health conditions (e.g., chronic stress, burnout, arrhythmia, ectopic beats, pregnancy-related complications, and the like). In such cases, the system may identify a quantity and/or frequency of spikes in the user's variability metric curve 405 and determine that the quantity and/or frequency of spikes in the user's variability metric curve 405 may exceed a threshold, thereby indicating an underlying long-term physical and or mental health conditions.

For example, the system may determine a plurality of variability metrics (e.g., a quantity of peaks in the variability metric curve 405, a frequency of peaks in the variability metric curve 405) associated with a plurality of sleep periods of the user. The system may identify that a subset of variability metrics exceeds a threshold metric. An alert may be provided to the user, as described with reference to FIG. 5. The alerts may be associated with burnout, chronic stress, mental health conditions, and the like. In such cases, the system may generate an alert provided to the user based on a quantity of the subset of variability metrics exceeding a threshold quantity, a frequency of the subset of variability metrics exceeding a threshold frequency, or both.

In some cases, the HRV data and/or the variability metrics of the user (as represented by the variability metric curve 405) may be used to determine various physiological parameters using a machine learning model. The machine learning model may be an example of a mathematical model comprising at least one equation, statistical or machine learning algorithm, a machine learning classifier, machine learning algorithm, a non-classifier mathematical mode, algorithm-based techniques, or the like.

In some examples, the HRV data and/or the variability metrics (e.g., variability metric curve 405) may be used as inputs in machine learning models to calculate one or more physiological parameters, adjust operational parameters of the wearable device 104, adjust the user's surroundings, or any combination thereof. The physiological parameters may include, but are not limited to, a sleep staging metric, a Readiness Score, a recovery metric, a stress metric, a pregnancy-related metric, an abnormality in physiological function metric, or a combination thereof. For example, the system may generate, via the machine learning model, the one or more physiological parameters of the user based on inputting the variability metric 405 into the machine learning model.

The system may calculate the physiological parameters based on the weights applied. In some cases, the HRV data may be weighted according to the user's variability metric(s). For example, the system may weight the HRV data differently based on whether or not the variability metric (e.g., value of the variability metric curve 405) satisfies certain thresholds. In such cases, the machine learning model may be configured to weight the plurality of HRV values in accordance with a first predictive weight of the one or more predictive weights based on the variability metric 405 satisfying a threshold value or a second predictive weight of the one or more predictive weights based on the variability metric (e.g., value of the variability metric curve 405) failing to satisfy threshold value. The one or more physiological parameters of the user are calculated based on weighting the plurality of HRV values in accordance with one of the first predictive weight or the second predictive weight.

In some examples, the system may determine that the variability metric satisfies the threshold value. The variability metric satisfies the threshold value based on the variability metric being greater than the threshold value. In such cases, the machine learning model may be configured to weight the plurality of HRV values in accordance with the first predictive weight of the one or more predictive weights based on the variability metric satisfying the threshold value. The one or more physiological parameters of the user are calculated based on weighting the plurality of HRV values in accordance with the first predictive weight. In such cases, the HRV data is weighed less heavily when the variability metric 405 is higher. For example, the first predictive weight is less than the second predictive weight.

In some cases, the system may determine that the variability metric fails to satisfy the threshold value. The variability metric fails to satisfy the threshold value based on the variability metric being less than the threshold value. In such cases, the machine learning model may be configured to weight the plurality of HRV values in accordance with the second predictive weight of the one or more predictive weights based on the variability metric failing to satisfy the threshold value. The one or more physiological parameters of the user are calculated based on weighting the plurality of HRV values in accordance with the second predictive weight. In such cases, the HRV data is weighed more heavily when the variability metric is lower. For example, the second predictive weight is greater than the first predictive weight.

That is, high variability metrics (e.g., high values of the variability metric curve 405) indicate larger swings in the user's HRV data, and low variability metrics 405 indicate relatively constant (e.g., consistent) HRV data. When calculating the user's recovery metric, for example, HRV data may be weighted less when the variability metric is high (e.g., indicating that the HRV data may be relatively unreliable), whereas HRV data may be weighted more heavily when the variability metric 405 is low (e.g., indicating relatively constant HRV data). In such cases, when calculating the user's physiological parameters, the HRV data may be weighted less heavily on days when the third event 410-c occurred and/or the fourth event 410-d occurred as indicated by a high variability metric. In other examples, when calculating the user's physiological parameters, the HRV data may be weighted more heavily on days when the first event 410-a occurred and/or the second event 410-b occurred as indicated by a low variability metric (e.g., low value of the variability metric curve 405).

The machine learning model may be continuously trained based on the machine learning model inputs and the machine learning model outputs. In such cases, the machine learning model may be continuously adjusted based on the HRV data, the variability metric, or both. For example, the system may train the machine learning model based on inputting the variability metric into the machine learning model and calculating the one or more physiological parameters of the user.

FIG. 5 shows an example of a GUI 500 that supports techniques for utilizing the variability of HRV in accordance with aspects of the present disclosure. Aspects of the GUI 500 may implement, or be implemented by, aspects of the system 100, system 200, timing diagram 300, timing diagram 400, or any combination thereof. For example, the GUI 500 may be an example of a GUI 275 of a user device 106 (e.g., user device 106 a, 106 b, 106 c) corresponding to a user 102.

In some examples, the GUI 500 illustrates a series of application pages 505 that may be displayed to a user via the GUI 500 (e.g., GUI 275 illustrated in FIG. 2). The server of the system may generate an alert 510, a message 520, an HRV card 515, or a combination thereof for display on the GUI 500 on a user device that indicates average HRV, the variability metric, one or more calculated physiological parameters, a recommendation for one or more actions to be taken by the user to improve the variability metric of the HRV data, and the like.

For example, the server may cause the GUI 500 of the user device (e.g., mobile device) to display the HRV card 515 associated with the HRV data (e.g., via application page 505). In such cases, the system may output the HRV data on the GUI 500 of the user device. Continuing with the example above, the user may be presented with the application page 505 upon opening the wearable application. In such cases, the application page 505 may include the HRV card 515 on the home page.

In some implementations, the application page 505 may notify the user of the HRV data, the one or more physiological parameters of the user, the recommendation, or a combination thereof and/or prompt the user to perform a variety of tasks in the activity GUI. The notifications and prompts may include text, graphics, and/or other user interface elements. The user device may display notifications and prompts in a separate window on the home screen and/or overlaid onto other screens (e.g., at the top of the home screen). In some cases, the user device may display the notifications and prompts on a mobile device, a user's watch device, or both. Additionally, or alternatively, as described herein, the system may automatically adjust operational parameters of the wearable device and/or adjust characteristics of the user's surrounding environment (e.g., adjust parameters of external devices that are able to control characteristics of the user's environment) based on the determined HRV data and/or variability metric.

In some examples, the application page 505 may display one or more calculated physiological parameters and a recommendation for one or more actions to be taken by the user to improve the variability metric of the HRV data via the message 520. In such cases, the application page 505 may include the message 520 on the home page. For example, the user may receive message 520, that may indicate the one or more calculated physiological parameters and a recommendation for one or more actions to be taken by the user to improve the variability metric of the HRV data. The messages 520 may be configurable/customizable, such that the user may receive different messages 520 based on the HRV data, the variability metric of the HRV data, or both, as described previously herein. Additionally, or alternatively, the system may proactively transmit signals to control external devices to implement such recommendations (e.g., transmit a signal to a smart thermostat to adjust the temperature of the user's bedroom at night). In such cases, the messages 520 may instead indicate to the user what adjustments have been made, why the adjustments were made, etc.

As shown in FIG. 5, the application page 505 may display the one or more calculated physiological parameters and a recommendation for one or more actions to be taken by the user to improve the variability metric of the HRV data via alert 510. The user may receive alert 510, which may prompt the user to verify receipt of the one or more calculated physiological parameters and the recommendation. In such cases, the application page 505 may prompt the user to confirm or dismiss the one or more calculated physiological parameters and the recommendation. The alert 510 may be generated and displayed based on the one or more physiological parameters and based on weighting the HRV data in accordance with one or more predictive weights that are based on the variability metric.

In some cases, the alert 510 may be provided to the user based on a quantity of a subset of variability metrics exceeding a threshold quantity, a frequency of the subset of variability metrics exceeding a threshold frequency, or both. For example, repeated spikes in variability or constantly elevated variability may be used to notify the user of underlying long-term physical and/or mental health conditions, including but not limited to, chronic stress and burnout. In such cases, the system may identify that the quantity and/or frequency of spikes in the user's HRV variability metrics exceeds a threshold and provide the alert 510 in response to the identification. The alert 510 may be associated with burnout, chronic stress, mental health conditions, and the like.

Additionally, in some implementations, the application page 505 may display one or more scores (e.g., Sleep Score, Readiness Score, Activity Score, etc.) for the user for the respective day. Moreover, in some cases, the variability metric of the HRV data may be used to update (e.g., modify) one or more scores associated with the user (e.g., Sleep Score, Readiness Score, etc.). That is, data associated with the variability metric of the HRV data may be used to update the scores for the user for the following calendar days. In such cases, the system may notify the user of the score update via alert 510.

The application page 505 may indicate one or more parameters, including a temperature, PPG, heart rate, HRV, respiratory rate, sleep data, blood oxygen data, and the like used to determine the variability metric via the HRV card 515. For example, the system may provide, via HRV card 515, the user with a graphical representation that illustrates the HRV data associated with the time intervals of the sleep period. For example, the HRV card 515 may include the HRV data associated with the time intervals of the sleep period for the user's previous night's sleep.

In some cases, the user may log tags via user input 525. For example, the system may receive user input 525 (e.g., tags) to log indications that may factor into the training classifiers, calculating the physiological parameters, providing the recommendation for one or more actions to be taken by the user to improve the variability metric of the HRV data, and the like. For example, the system may receive, via the GUI 500, the user input 525 including an indication of a rest day, an indication of an activity, an indication of a menstrual cycle onset day, one or more tags, or a combination thereof.

In some examples, the system may transmit one or more additional signals to the user device to cause the user device to display a prompt for the user to confirm a stress-inducing event. For example, the user device may display a prompt via alert 510 or message 520 indicating “Looks like your variability metric is above your average. Did you have a stressful day?” In such cases, the system may receive user input 525 confirming the stress-inducing event. In some implementations, the system may be configured to receive user inputs 525 regarding the stress-inducing event, for example, in order to train classifiers (e.g., supervised learning for a machine learning model) and improve identifying stress-inducing events based on HRV data. For example, the user device may receive user inputs 525, and these user inputs 525 may then be input into the classifier to train the classifier. In other examples, the user device may receive physiological data, from the wearable device, and the physiological data may then be input into the classifier to train the classifier.

In some examples, the system may transmit one or more additional signals to the user device that cause the user device to display a prompt for the user to confirm a non-stress-inducing event. For example, the user device may display a prompt alert 510 or message 520 indicating “Looks like your variability metric is below your average. Did you have a relaxing day?” The system may receive, via the user device, a confirmation of the non-stress-inducing event. The machine learning model may be retrained to identify non-stress-inducing events for the user based on the HRV data measured from the user and based on inputting the non-stress-inducing events and the confirmation into the machine learning model.

In some aspects, the application page 505 may include instructions for the user to modify one or more behaviors of the user to adjust (e.g., improve) their variability metric. In other words, the system may provide insights as to how the user can improve their variability metric. For example, the system may suggest that the user take a nap, take a walk, or go for a bike ride to improve their variability metric. In some cases, recommendations/insights provided by the system may be based on previous data acquired from the user. For instance, the system may recognize that the user's variability metric improved when the user went for a walk in the past, and may therefore suggest that the user go for a walk based on this previously-identified relationship.

In other words, the system may determine when the user took a walk in the past based on tags inputted by the user, based on an activity logged in a third-party application, based on activity detection performed by the wearable device or another component of the system. In such cases, the system may further recognize that the user's variability metric improved when the user took walks in the past. By way of another example, the system may determine that the user's variability metric improved when the user participated in a guided meditation within the wearable application or another third-party application.

In some cases, the user's logged symptoms (e.g., tags) in combination with the user's physiological data may characterize the HRV data, the variability metric, or both. In such cases, the user's logged user input 525 may contribute to calculating the one or more physiological parameters of the user based on weighting the HRV data in accordance with one or more predictive weights that are based on the variability metric. The logged user input 525 may be an example of an indication of a rest day, an indication of an activity target, an indication of a menstrual cycle onset day, one or more tags, or a combination thereof.

In some aspects, the user device may display instructions for the user to modify one or more behaviors of the user to adjust (e.g., improve) their variability metric. In other words, the system may provide insights as to how the user can improve their variability metric going forward to prevent burnout and/or chronic stress conditions, better cope with and/or recover from stress, and the like, for example. The users may be given insights, via messages 520, HRV card 515, and/or alerts 510 for possible causes of stress and how the user can better cope with and/or recover from stress.

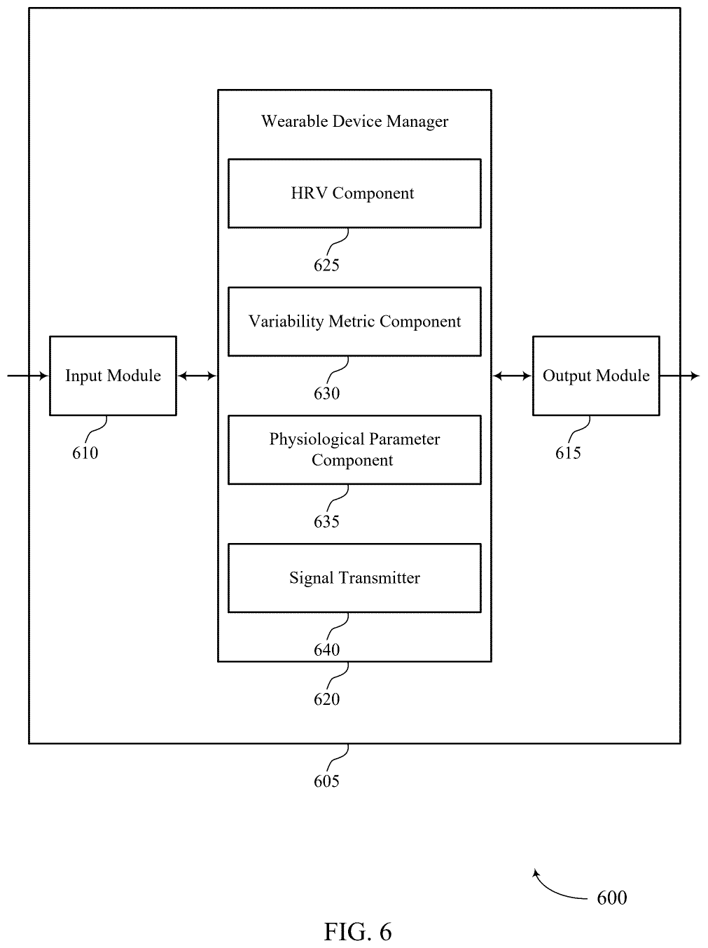

FIG. 6 shows a block diagram 600 of a device 605 that supports techniques for utilizing the variability of HRV in accordance with aspects of the present disclosure. The device 605 may include an input module 610, an output module 615, and a wearable device manager 620. The device 605, or one or more components of the device 605 (e.g., the input module 610, the output module 615, the wearable device manager 620), may include at least one processor, which may be coupled with at least one memory, to support the described techniques. Each of these components may be in communication with one another (e.g., via one or more buses).

For example, the wearable device manager 620 may include an HRV component 625, a variability metric component 630, a physiological parameter component 635, a signal transmitter 640, or any combination thereof. In some examples, the wearable device manager 620, or various components thereof, may be configured to perform various operations (e.g., receiving, monitoring, transmitting) using or otherwise in cooperation with the input module 610, the output module 615, or both. For example, the wearable device manager 620 may receive information from the input module 610, send information to the output module 615, or be integrated in combination with the input module 610, the output module 615, or both to receive information, transmit information, or perform various other operations as described herein.

The HRV component 625 may be configured as or otherwise support a means for identifying a plurality of HRV values associated with a plurality of time intervals of a sleep period based at least in part on HRV data collected via a wearable device throughout the sleep period. The variability metric component 630 may be configured as or otherwise support a means for determining a variability metric associated with the plurality of HRV values of the sleep period, the variability metric associated with one or more changes in the plurality of HRV values throughout the sleep period. The physiological parameter component 635 may be configured as or otherwise support a means for inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric. The signal transmitter 640 may be configured as or otherwise support a means for transmitting, using the one or more processors, one or more signals to a user device, the one or more signals comprising an instruction for a GUI) of the user device to display the one or more calculated physiological parameters and a recommendation for one or more actions to be taken by the user to improve the variability metric of the HRV data.

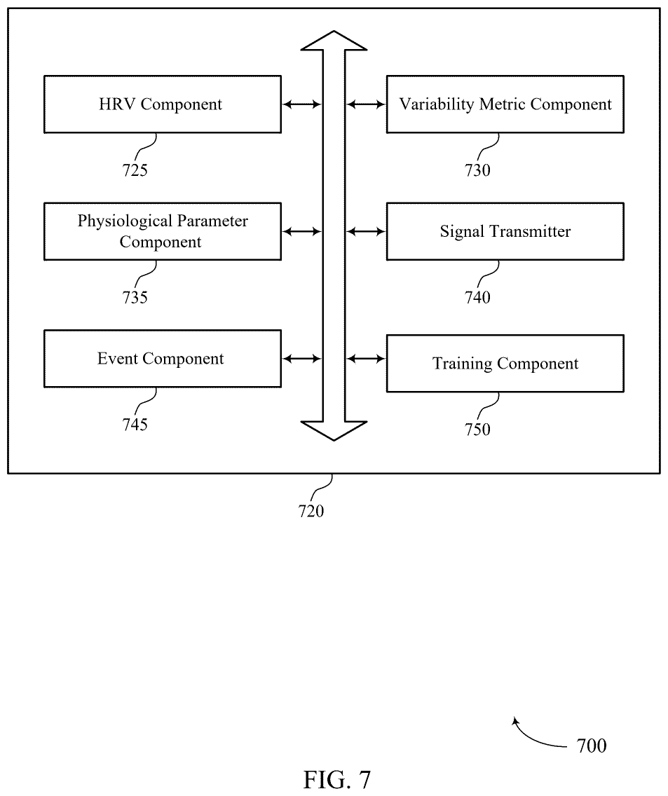

FIG. 7 shows a block diagram 700 of a wearable device manager 720 that supports techniques for utilizing the variability of HRV in accordance with aspects of the present disclosure. The wearable device manager 720 may be an example of aspects of a wearable device manager or a wearable device manager 620, or both, as described herein. The wearable device manager 720, or various components thereof, may be an example of means for performing various aspects of techniques for utilizing the variability of HRV as described herein. For example, the wearable device manager 720 may include an HRV component 725, a variability metric component 730, a physiological parameter component 735, a signal transmitter 740, an event component 745, a training component 750, or any combination thereof. Each of these components, or components of subcomponents thereof (e.g., one or more processors, one or more memories), may communicate, directly or indirectly, with one another (e.g., via one or more buses).

The HRV component 725 may be configured as or otherwise support a means for identifying a plurality of HRV values associated with a plurality of time intervals of a sleep period based at least in part on HRV data collected via a wearable device throughout the sleep period. The variability metric component 730 may be configured as or otherwise support a means for determining a variability metric associated with the plurality of HRV values of the sleep period, the variability metric associated with one or more changes in the plurality of HRV values throughout the sleep period. The physiological parameter component 735 may be configured as or otherwise support a means for inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric. The signal transmitter 740 may be configured as or otherwise support a means for transmitting, using the one or more processors, one or more signals to a user device, the one or more signals comprising an instruction for a GUI of the user device to display the one or more calculated physiological parameters and a recommendation for one or more actions to be taken by the user to improve the variability metric of the HRV data.

In some examples, the physiological parameter component 735 may be configured as or otherwise support a means for generating, via the machine learning model, the one or more physiological parameters of the user based at least in part on inputting the variability metric into the machine learning model, wherein the one or more physiological parameters comprises a sleep staging metric, a Readiness Score, a recovery metric, a stress metric (e.g., Stress Score), a pregnancy-related metric, or a combination thereof.

In some examples, the machine learning model is configured to weight the plurality of HRV values in accordance with a first predictive weight of the one or more predictive weights based at least in part on the variability metric satisfying a threshold value. In some examples, the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

In some examples, the second predictive weight is greater than the first predictive weight. In some examples, the variability metric satisfies the threshold value based at least in part on the variability metric being greater than the threshold value.

In some examples, to support determining the variability metric, the variability metric component 730 may be configured as or otherwise support a means for determining a difference between a first percentile of the plurality of HRV values and a second percentile of the plurality of HRV values based at least in part on identifying the plurality of HRV values, wherein the variability metric associated with the plurality of HRV values is based at least in part on the difference.

In some examples, to support determining the variability metric, the variability metric component 730 may be configured as or otherwise support a means for determining a standard deviation of the plurality of HRV values based at least in part on identifying the plurality of HRV values, wherein the variability metric associated with the plurality of HRV values is based at least in part on the standard deviation.

In some examples, to support determining the variability metric, the variability metric component 730 may be configured as or otherwise support a means for determining a coefficient of variation for the plurality of HRV values based at least in part on determining the standard deviation, wherein the variability metric associated with the plurality of HRV values is based at least in part on the coefficient of variation.

In some examples, the event component 745 may be configured as or otherwise support a means for identifying a stress-inducing event experienced by the user during a time interval preceding the sleep period based at least in part on the variability metric exceeding a threshold value, wherein the one or more signals are configured to cause the user device to display an indication of the stress-inducing event.

In some examples, the signal transmitter 740 may be configured as or otherwise support a means for transmitting one or more additional signals to the user device, the one or more additional signals configured to cause the user device to display a prompt for the user to confirm the stress-inducing event. In some examples, the event component 745 may be configured as or otherwise support a means for receiving, via the user device, a confirmation of the stress-inducing event. In some examples, the training component 750 may be configured as or otherwise support a means for retraining the machine learning model to identify stress-inducing events for the user based on the HRV data measured from the user based at least in part on inputting the stress-inducing event and the confirmation into the machine learning model.

In some examples, the training component 750 may be configured as or otherwise support a means for training the machine learning model based at least in part on inputting the variability metric into the machine learning model and calculating the one or more physiological parameters of the user.

In some examples, the training component 750 may be configured as or otherwise support a means for training the machine learning model based on a plurality of features within a training physiological dataset associated with a plurality of users, the training physiological dataset comprising a plurality of HRV values associated with the plurality of users.

In some examples, the variability metric component 730 may be configured as or otherwise support a means for determining a plurality of variability metrics associated with a plurality of sleep periods of the user, wherein the variability metric is included within the plurality of variability metrics. In some examples, the variability metric component 730 may be configured as or otherwise support a means for identifying a subset of variability metrics that exceed a threshold metric. In some examples, the event component 745 may be configured as or otherwise support a means for generating an alert provided to the user based at least in part on a quantity of the subset of variability metrics exceeding a threshold quantity, a frequency of the subset of variability metrics exceeding a threshold frequency, or both.

In some examples, the wearable device comprises a wearable ring device.

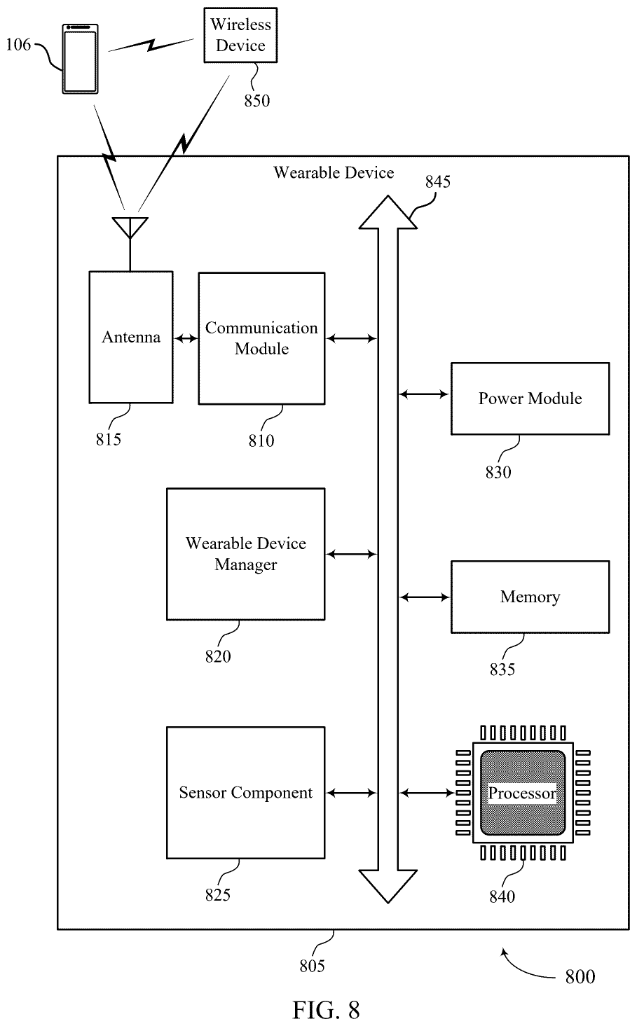

FIG. 8 shows a diagram of a system 800 including a device 805 that supports techniques for utilizing the variability of HRV in accordance with aspects of the present disclosure. The device 805 may be an example of or include components of a device 605 as described herein. The device 805 may include an example of a wearable device 104, as described previously herein. The device 805 may include components for bi-directional communications including components for transmitting and receiving communications with a user device 106 and a server 110, such as a wearable device manager 820, a communication module 810, one or more antennas 815, a sensor component 825, a power module 830, at least one memory 835, at least one processor 840, and a wireless device 850. These components may be in electronic communication or otherwise coupled (e.g., operatively, communicatively, functionally, electronically, electrically) via one or more buses (e.g., a bus 845).

For example, the wearable device manager 820 may be configured as or otherwise support a means for identifying a plurality of HRV values associated with a plurality of time intervals of a sleep period based at least in part on HRV data collected via a wearable device throughout the sleep period. The wearable device manager 820 may be configured as or otherwise support a means for determining a variability metric associated with the plurality of HRV values of the sleep period, the variability metric associated with one or more changes in the plurality of HRV values throughout the sleep period. The wearable device manager 820 may be configured as or otherwise support a means for inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric. The wearable device manager 820 may be configured as or otherwise support a means for transmitting, using the one or more processors, one or more signals to a user device, the one or more signals comprising an instruction for a GUI of the user device to display the one or more calculated physiological parameters and a recommendation for one or more actions to be taken by the user to improve the variability metric of the HRV data.

By including or configuring the wearable device manager 820 in accordance with examples as described herein, the device 805 may support techniques for improved communication reliability, reduced latency, improved user experience related to reduced processing, reduced power consumption, more efficient utilization of communication resources, improved coordination between devices, longer battery life, improved utilization of processing capability, and the like.

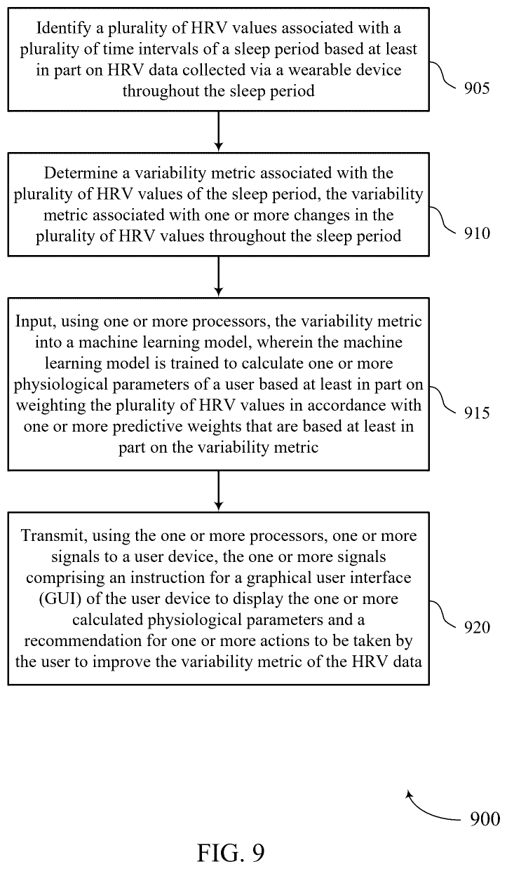

FIG. 9 shows a flowchart illustrating a method 900 that supports techniques for utilizing the variability of HRV in accordance with aspects of the present disclosure. The operations of the method 900 may be implemented by a wearable device or its components as described herein. For example, the operations of the method 900 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 905, the method may include identifying a plurality of HRV values associated with a plurality of time intervals of a sleep period based at least in part on HRV data collected via a wearable device throughout the sleep period. The operations of 905 may be performed in accordance with examples as disclosed herein. In some examples, aspects of the operations of 905 may be performed by an HRV component 725 as described with reference to FIG. 7.

At 910, the method may include determining a variability metric associated with the plurality of HRV values of the sleep period, the variability metric associated with one or more changes in the plurality of HRV values throughout the sleep period. The operations of 910 may be performed in accordance with examples as disclosed herein. In some examples, aspects of the operations of 910 may be performed by a variability metric component 730 as described with reference to FIG. 7.

At 915, the method may include inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric. The operations of 915 may be performed in accordance with examples as disclosed herein. In some examples, aspects of the operations of 915 may be performed by a physiological parameter component 735 as described with reference to FIG. 7.

At 920, the method may include transmitting, using the one or more processors, one or more signals to a user device, the one or more signals comprising an instruction for a GUI of the user device to display the one or more calculated physiological parameters and a recommendation for one or more actions to be taken by the user to improve the variability metric of the HRV data. The operations of 920 may be performed in accordance with examples as disclosed herein. In some examples, aspects of the operations of 920 may be performed by a signal transmitter 740 as described with reference to FIG. 7.

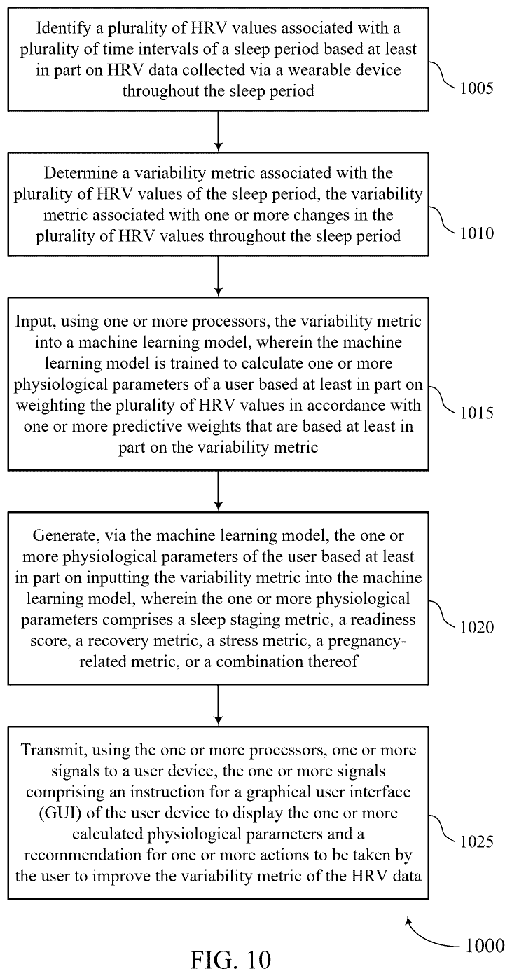

FIG. 10 shows a flowchart illustrating a method 1000 that supports techniques for utilizing the variability of HRV in accordance with aspects of the present disclosure. The operations of the method 1000 may be implemented by a wearable device or its components as described herein. For example, the operations of the method 1000 may be performed by a wearable device as described with reference to FIGS. 1 through 8. In some examples, a wearable device may execute a set of instructions to control the functional elements of the wearable device to perform the described functions. Additionally, or alternatively, the wearable device may perform aspects of the described functions using special-purpose hardware.

At 1005, the method may include identifying a plurality of HRV values associated with a plurality of time intervals of a sleep period based at least in part on HRV data collected via a wearable device throughout the sleep period. The operations of 1005 may be performed in accordance with examples as disclosed herein. In some examples, aspects of the operations of 1005 may be performed by an HRV component 725 as described with reference to FIG. 7.

At 1010, the method may include determining a variability metric associated with the plurality of HRV values of the sleep period, the variability metric associated with one or more changes in the plurality of HRV values throughout the sleep period. The operations of 1010 may be performed in accordance with examples as disclosed herein. In some examples, aspects of the operations of 1010 may be performed by a variability metric component 730 as described with reference to FIG. 7.

At 1015, the method may include inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric. The operations of 1015 may be performed in accordance with examples as disclosed herein. In some examples, aspects of the operations of 1015 may be performed by a physiological parameter component 735 as described with reference to FIG. 7.

At 1020, the method may include generating, via the machine learning model, the one or more physiological parameters of the user based at least in part on inputting the variability metric into the machine learning model, wherein the one or more physiological parameters comprises a sleep staging metric (e.g., Sleep Score), a Readiness Score, a recovery metric, a stress metric, a pregnancy-related metric, or a combination thereof. The operations of 1025 may be performed in accordance with examples as disclosed herein. In some examples, aspects of the operations of 1025 may be performed by a physiological parameter component 735 as described with reference to FIG. 7.

At 1025, the method may include transmitting, using the one or more processors, one or more signals to a user device, the one or more signals comprising an instruction for a GUI of the user device to display the one or more calculated physiological parameters and a recommendation for one or more actions to be taken by the user to improve the variability metric of the HRV data. The operations of 1020 may be performed in accordance with examples as disclosed herein. In some examples, aspects of the operations of 1020 may be performed by a signal transmitter 740 as described with reference to FIG. 7.

It should be noted that the methods described above describe possible implementations, and that the operations and the steps may be rearranged or otherwise modified and that other implementations are possible. Furthermore, aspects from two or more of the methods may be combined.

A system is described. The system may include a wearable device configured to measure physiological data from a user via one or more light-emitting components and one or more light-receiving components of the wearable device, the physiological data comprising at least HRV data measured continuously from the user throughout a plurality of time intervals of a sleep period that the user is asleep, a user device communicatively coupled with the wearable device, and one or more processors communicatively coupled with the wearable device and the user device, the one or more processors configured to identify a plurality of HRV values associated with the plurality of time intervals of the sleep period based at least in part on the HRV data collected via the wearable device throughout the sleep period, determine a variability metric associated with the plurality of HRV values of the sleep period, the variability metric associated with one or more changes in the plurality of HRV values throughout the sleep period, input, using the one or more processors, the variability metric into a machine learning model, wherein the machine learning model is trained to calculate one or more physiological parameters of the user based at least in part on weighting the plurality of HRV values in accordance with one or more predictive weights that are based at least in part on the variability metric, and transmit, using the one or more processors, one or more signals to the user device, the one or more signals comprising an instruction for a GUI of the user device to display the one or more calculated physiological parameters and a recommendation for one or more actions to be taken by the user to improve the variability metric of the HRV data

A method is described. The method may include identifying a plurality of HRV values associated with a plurality of time intervals of a sleep period based at least in part on HRV data collected via a wearable device throughout the sleep period, determining a variability metric associated with the plurality of HRV values of the sleep period, the variability metric associated with one or more changes in the plurality of HRV values throughout the sleep period, inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric, and transmitting, using the one or more processors, one or more signals to a user device, the one or more signals comprising an instruction for a GUI of the user device to display the one or more calculated physiological parameters and a recommendation for one or more actions to be taken by the user to improve the variability metric of the HRV data.

An apparatus is described. The apparatus may include one or more memories storing processor executable code, and one or more processors coupled with the one or more memories. The one or more processors may individually or collectively be operable to execute the code to cause the apparatus to identify a plurality of HRV values associated with a plurality of time intervals of a sleep period based at least in part on HRV data collected via a wearable device throughout the sleep period, determine a variability metric associated with the plurality of HRV values of the sleep period, the variability metric associated with one or more changes in the plurality of HRV values throughout the sleep period, input, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric, and transmit, using the one or more processors, one or more signals to a user device, the one or more signals comprising an instruction for a GUI of the user device to display the one or more calculated physiological parameters and a recommendation for one or more actions to be taken by the user to improve the variability metric of the HRV data.

Another apparatus is described. The apparatus may include means for identifying a plurality of HRV values associated with a plurality of time intervals of a sleep period based at least in part on HRV data collected via a wearable device throughout the sleep period, means for determining a variability metric associated with the plurality of HRV values of the sleep period, the variability metric associated with one or more changes in the plurality of HRV values throughout the sleep period, means for inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric, and means for transmitting, using the one or more processors, one or more signals to a user device, the one or more signals comprising an instruction for a GUI of the user device to display the one or more calculated physiological parameters and a recommendation for one or more actions to be taken by the user to improve the variability metric of the HRV data.

A non-transitory computer-readable medium storing code is described. The code may include instructions executable by one or more processors to identify a plurality of HRV values associated with a plurality of time intervals of a sleep period based at least in part on HRV data collected via a wearable device throughout the sleep period, determine a variability metric associated with the plurality of HRV values of the sleep period, the variability metric associated with one or more changes in the plurality of HRV values throughout the sleep period, input, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric, and transmit, using the one or more processors, one or more signals to a user device, the one or more signals comprising an instruction for a GUI of the user device to display the one or more calculated physiological parameters and a recommendation for one or more actions to be taken by the user to improve the variability metric of the HRV data.

Some examples of the system, method, apparatus, and non-transitory computer-readable medium described herein may further include operations, features, means, or instructions for generating, via the machine learning model, the one or more physiological parameters of the user based at least in part on inputting the variability metric into the machine learning model, wherein the one or more physiological parameters comprises a sleep staging metric (e.g., Sleep Score), a Readiness Score, a recovery metric, a stress metric, a pregnancy-related metric, or a combination thereof.

In some examples of the system, method, apparatus, and non-transitory computer-readable medium described herein, the machine learning model may be configured to weight the plurality of HRV values in accordance with a first predictive weight of the one or more predictive weights based at least in part on the variability metric satisfying a threshold value and the machine learning model may be configured to weight the plurality of HRV values in accordance with a second predictive weight of the one or more predictive weights based at least in part on the variability metric failing to satisfy the threshold value, wherein the one or more physiological parameters of the user may be calculated based at least in part on weighting the plurality of HRV values in accordance with one of the first predictive weight or the second predictive weight.

In some examples of the system method, apparatus, and non-transitory computer-readable medium described herein, the second predictive weight may be greater than the first predictive weight and the variability metric satisfies the threshold value based at least in part on the variability metric being greater than the threshold value.

In some examples of the system, method, apparatus, and non-transitory computer-readable medium described herein, determining the variability metric may include operations, features, means, or instructions for determining a difference between a first percentile of the plurality of HRV values and a second percentile of the plurality of HRV values based at least in part on identifying the plurality of HRV values, wherein the variability metric associated with the plurality of HRV values may be based at least in part on the difference.

In some examples of the system, method, apparatus, and non-transitory computer-readable medium described herein, determining the variability metric may include operations, features, means, or instructions for determining a standard deviation of the plurality of HRV values based at least in part on identifying the plurality of HRV values, wherein the variability metric associated with the plurality of HRV values may be based at least in part on the standard deviation.

In some examples of the system, method, apparatus, and non-transitory computer-readable medium described herein, determining the variability metric may include operations, features, means, or instructions for determining a coefficient of variation for the plurality of HRV values based at least in part on determining the standard deviation, wherein the variability metric associated with the plurality of HRV values may be based at least in part on the coefficient of variation.

Some examples of the system, method, apparatus, and non-transitory computer-readable medium described herein may further include operations, features, means, or instructions for identifying a stress-inducing event experienced by the user during a time interval preceding the sleep period based at least in part on the variability metric exceeding a threshold value, wherein the one or more signals may be configured to cause the user device to display an indication of the stress-inducing event.

Some examples of the system, method, apparatus, and non-transitory computer-readable medium described herein may further include operations, features, means, or instructions for transmitting one or more additional signals to the user device, the one or more additional signals configured to cause the user device to display a prompt for the user to confirm the stress-inducing event, receiving, via the user device, a confirmation of the stress-inducing event, and retraining the machine learning model to identify stress-inducing events for the user based on the HRV data measured from the user based at least in part on inputting the stress-inducing event and the confirmation into the machine learning model.

Some examples of the system, method, apparatus, and non-transitory computer-readable medium described herein may further include operations, features, means, or instructions for training the machine learning model based at least in part on inputting the variability metric into the machine learning model and calculating the one or more physiological parameters of the user.

Some examples of the system, method, apparatus, and non-transitory computer-readable medium described herein may further include operations, features, means, or instructions for training the machine learning model based on a plurality of features within a training physiological dataset associated with a plurality of users, the training physiological dataset comprising a plurality of HRV values associated with the plurality of users.

Some examples of the system, method, apparatus, and non-transitory computer-readable medium described herein may further include operations, features, means, or instructions for determining a plurality of variability metrics associated with a plurality of sleep periods of the user, wherein the variability metric may be included within the plurality of variability metrics, identifying a subset of variability metrics that exceed a threshold metric, and generating an alert provided to the user based at least in part on a quantity of the subset of variability metrics exceeding a threshold quantity, a frequency of the subset of variability metrics exceeding a threshold frequency, or both.

In some examples of the system, method, apparatus, and non-transitory computer-readable medium described herein, the wearable device comprises a wearable ring device.

Aspects of the present disclosure are set out below. Each item below corresponds to an aspect of the disclosure, and each possible combination of any of the items corresponds to an aspect of the disclosure. When items are combined, two or more instances of an element with the same name are to be understood as referring to the same element, even if the element is introduced by the word “a” more than once. Each possible combination of any features from the items below with any features described above also forms part of the disclosure:

Aspect 1: A system, comprising: a wearable device configured to measure physiological data from a user via one or more light-emitting components and one or more light-receiving components of the wearable device, the physiological data comprising at least heart rate variability (HRV) data measured continuously from the user throughout a plurality of time intervals of a sleep period that the user is asleep; a user device communicatively coupled with the wearable device; and one or more processors communicatively coupled with the wearable device and the user device, the one or more processors configured to: identify a plurality of HRV values associated with the plurality of time intervals of the sleep period based at least in part on the HRV data collected via the wearable device throughout the sleep period; determine a variability metric associated with the plurality of HRV values of the sleep period, the variability metric associated with one or more changes in the plurality of HRV values throughout the sleep period; input, using the one or more processors, the variability metric into a machine learning model, wherein the machine learning model is trained to calculate one or more physiological parameters of the user based at least in part on weighting the plurality of HRV values in accordance with one or more predictive weights that are based at least in part on the variability metric; and transmit, using the one or more processors, one or more signals to the user device, the one or more signals comprising an instruction for a graphical user interface (GUI) of the user device to display the one or more calculated physiological parameters and a recommendation for one or more actions to be taken by the user to improve the variability metric of the HRV data.

Aspect 2: The system of aspect 1, wherein the one or more processors are further configured to: generate, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

Aspect 3: The system of aspect 1 through 2, wherein the machine learning model is configured to weight the plurality of HRV values in accordance with a first predictive weight of the one or more predictive weights based at least in part on the variability metric satisfying a threshold value, or wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

Aspect 4. The system of aspect 3, wherein the second predictive weight is greater than the first predictive weight, and the variability metric satisfies the threshold value based at least in part on the variability metric being greater than the threshold value.

Aspect 5: The system of aspect 1 through 4, wherein, to determine the variability metric, the one or more processors are further configured to: determine a difference between a first percentile of the plurality of HRV values and a second percentile of the plurality of HRV values based at least in part on identifying the plurality of HRV values, wherein the variability metric associated with the plurality of HRV values is based at least in part on the difference.

Aspect 6: The system of aspect 1 through 5, wherein, to determine the variability metric, the one or more processors are further configured to: determine a standard deviation of the plurality of HRV values based at least in part on identifying the plurality of HRV values, wherein the variability metric associated with the plurality of HRV values is based at least in part on the standard deviation.

Aspect 7: The system of aspect 6, wherein, to determine the variability metric, the one or more processors are further configured to: determine a coefficient of variation for the plurality of HRV values based at least in part on determining the standard deviation, wherein the variability metric associated with the plurality of HRV values is based at least in part on the coefficient of variation.

Aspect 8: The system of aspect 1 through 7, wherein the one or more processors are configured to: identify a stress-inducing event experienced by the user during a time interval preceding the sleep period based at least in part on the variability metric exceeding a threshold value, wherein the one or more signals are configured to cause the user device to display an indication of the stress-inducing event.

Aspect 9: The system of aspect 8, wherein the one or more processors are configured to: transmit one or more additional signals to the user device, the one or more additional signals configured to cause the user device to display a prompt for the user to confirm the stress-inducing event; receive, via the user device, a confirmation of the stress-inducing event; and retrain the machine learning model to identify stress-inducing events for the user based on the HRV data measured from the user based at least in part on inputting the stress-inducing event and the confirmation into the machine learning model.

Aspect 10: The system of aspect 1 through 9, wherein the one or more processors are configured to: train the machine learning model based at least in part on inputting the variability metric into the machine learning model and calculating the one or more physiological parameters of the user.

Aspect 11: The system of aspect 1 through 10, wherein the one or more processors are configured to: train the machine learning model based on a plurality of features within a training physiological dataset associated with a plurality of users, the training physiological dataset comprising a plurality of HRV values associated with the plurality of users.

Aspect 12: The system of aspect 1 through 11, wherein the one or more processors are configured to: determine a plurality of variability metrics associated with a plurality of sleep periods of the user, wherein the variability metric is included within the plurality of variability metrics; identify a subset of variability metrics that exceed a threshold metric; and generate an alert provided to the user based at least in part on a quantity of the subset of variability metrics exceeding a threshold quantity, a frequency of the subset of variability metrics exceeding a threshold frequency, or both.

Aspect 13: The system of aspect 1 through 12, wherein the wearable device comprises a wearable ring device.

Aspect 14: A method, comprising: identifying a plurality of HRV values associated with a plurality of time intervals of a sleep period based at least in part on HRV data collected via a wearable device throughout the sleep period; determining a variability metric associated with the plurality of HRV values of the sleep period, the variability metric associated with one or more changes in the plurality of HRV values throughout the sleep period; inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric; and transmitting, using the one or more processors, one or more signals to a user device, the one or more signals comprising an instruction for a graphical user interface (GUI) of the user device to display the one or more calculated physiological parameters and a recommendation for one or more actions to be taken by the user to improve the variability metric of the HRV data.

Aspect 15: The method of aspect 14, further comprising: generating, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

Aspect 16: The method of aspect 14 through 15, wherein the machine learning model is configured to weight the plurality of HRV values in accordance with a first predictive weight of the one or more predictive weights based at least in part on the variability metric satisfying a threshold value, or wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

Aspect 17: The method of aspect 16, wherein the second predictive weight is greater than the first predictive weight, and wherein the variability metric satisfies the threshold value based at least in part on the variability metric being greater than the threshold value.

Aspect 18: A non-transitory computer-readable medium storing code, the code comprising instructions executable by one or more processors to: identify a plurality of HRV values associated with a plurality of time intervals of a sleep period based at least in part on HRV data collected via a wearable device throughout the sleep period; determine a variability metric associated with the plurality of HRV values of the sleep period, the variability metric associated with one or more changes in the plurality of HRV values throughout the sleep period; input, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric; and transmit, using the one or more processors, one or more signals to a user device, the one or more signals comprising an instruction for a graphical user interface (GUI) of the user device to display the one or more calculated physiological parameters and a recommendation for one or more actions to be taken by the user to improve the variability metric of the HRV data.

Aspect 19: The non-transitory computer-readable medium of aspect 18, wherein the instructions are further executable by the one or more processors to: generate, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

Aspect 20: The non-transitory computer-readable medium of aspect 18 through 19, wherein the machine learning model is configured to weight the plurality of HRV values in accordance with a first predictive weight of the one or more predictive weights based at least in part on the variability metric satisfying a threshold value, or wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

Clause 1: A system, comprising: a wearable device configured to measure physiological data from a user via one or more light-emitting components and one or more light-receiving components of the wearable device, the physiological data comprising at least heart rate variability (HRV) data measured continuously from the user throughout a plurality of time intervals of a sleep period that the user is asleep; a user device communicatively coupled with the wearable device; and one or more processors communicatively coupled with the wearable device and the user device, the one or more processors configured to: identify a plurality of HRV values associated with the plurality of time intervals of the sleep period based at least in part on the HRV data collected via the wearable device throughout the sleep period; determine a variability metric associated with the plurality of HRV values of the sleep period, the variability metric associated with one or more changes in the plurality of HRV values throughout the sleep period; input, using the one or more processors, the variability metric into a machine learning model, wherein the machine learning model is trained to calculate one or more physiological parameters of the user based at least in part on weighting the plurality of HRV values in accordance with one or more predictive weights that are based at least in part on the variability metric; and transmit, using the one or more processors, one or more signals to the user device, the one or more signals comprising an instruction for a graphical user interface (GUI) of the user device to display the one or more calculated physiological parameters and a recommendation for one or more actions to be taken by the user to improve the variability metric of the HRV data.

Clause 2: The system of clause 1, wherein the one or more processors are further configured to: generate, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

Clause 3: The system of any of clauses 1-2, wherein the machine learning model is configured to weight the plurality of HRV values in accordance with a first predictive weight of the one or more predictive weights based at least in part on the variability metric satisfying a threshold value, or wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

Clause 4. The system of clause 3, wherein the second predictive weight is greater than the first predictive weight, and the variability metric satisfies the threshold value based at least in part on the variability metric being greater than the threshold value.

Clause 5: The system of any of clauses 1-4, wherein, to determine the variability metric, the one or more processors are further configured to: determine a difference between a first percentile of the plurality of HRV values and a second percentile of the plurality of HRV values based at least in part on identifying the plurality of HRV values, wherein the variability metric associated with the plurality of HRV values is based at least in part on the difference.

Clause 6: The system of any of clauses 1-5, wherein, to determine the variability metric, the one or more processors are further configured to: determine a standard deviation of the plurality of HRV values based at least in part on identifying the plurality of HRV values, wherein the variability metric associated with the plurality of HRV values is based at least in part on the standard deviation.

Clause 7: The system of clause 6, wherein, to determine the variability metric, the one or more processors are further configured to: determine a coefficient of variation for the plurality of HRV values based at least in part on determining the standard deviation, wherein the variability metric associated with the plurality of HRV values is based at least in part on the coefficient of variation.

Clause 8: The system of any of clauses 1-7, wherein the one or more processors are configured to: identify a stress-inducing event experienced by the user during a time interval preceding the sleep period based at least in part on the variability metric exceeding a threshold value, wherein the one or more signals are configured to cause the user device to display an indication of the stress-inducing event.

Clause 9: The system of clause 8, wherein the one or more processors are configured to: transmit one or more additional signals to the user device, the one or more additional signals configured to cause the user device to display a prompt for the user to confirm the stress-inducing event; receive, via the user device, a confirmation of the stress-inducing event; and retrain the machine learning model to identify stress-inducing events for the user based on the HRV data measured from the user based at least in part on inputting the stress-inducing event and the confirmation into the machine learning model.

Clause 10: The system of any of clauses 1-9, wherein the one or more processors are configured to: train the machine learning model based at least in part on inputting the variability metric into the machine learning model and calculating the one or more physiological parameters of the user.

Clause 11: The system of any of clauses 1-10, wherein the one or more processors are configured to: train the machine learning model based on a plurality of features within a training physiological dataset associated with a plurality of users, the training physiological dataset comprising a plurality of HRV values associated with the plurality of users.

Clause 12: The system of any of clauses 1-11, wherein the one or more processors are configured to: determine a plurality of variability metrics associated with a plurality of sleep periods of the user, wherein the variability metric is included within the plurality of variability metrics; identify a subset of variability metrics that exceed a threshold metric; and generate an alert provided to the user based at least in part on a quantity of the subset of variability metrics exceeding a threshold quantity, a frequency of the subset of variability metrics exceeding a threshold frequency, or both.

Clause 13: The system of any of clauses 1-12, wherein the wearable device comprises a wearable ring device.

Clause 14: A method, comprising: identifying a plurality of HRV values associated with a plurality of time intervals of a sleep period based at least in part on HRV data collected via a wearable device throughout the sleep period; determining a variability metric associated with the plurality of HRV values of the sleep period, the variability metric associated with one or more changes in the plurality of HRV values throughout the sleep period; inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric; and transmitting, using the one or more processors, one or more signals to a user device, the one or more signals comprising an instruction for a graphical user interface (GUI) of the user device to display the one or more calculated physiological parameters and a recommendation for one or more actions to be taken by the user to improve the variability metric of the HRV data.

Clause 15: The method of clause 14, further comprising: generating, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

Clause 16: The method of any of clauses 14-15, wherein the machine learning model is configured to weight the plurality of HRV values in accordance with a first predictive weight of the one or more predictive weights based at least in part on the variability metric satisfying a threshold value, or wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

Clause 17: The method of clause 16, wherein the second predictive weight is greater than the first predictive weight, and wherein the variability metric satisfies the threshold value based at least in part on the variability metric being greater than the threshold value.

Clause 18: A non-transitory computer-readable medium storing code, the code comprising instructions executable by one or more processors to: identify a plurality of HRV values associated with a plurality of time intervals of a sleep period based at least in part on HRV data collected via a wearable device throughout the sleep period; determine a variability metric associated with the plurality of HRV values of the sleep period, the variability metric associated with one or more changes in the plurality of HRV values throughout the sleep period; input, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric; and transmit, using the one or more processors, one or more signals to a user device, the one or more signals comprising an instruction for a graphical user interface (GUI) of the user device to display the one or more calculated physiological parameters and a recommendation for one or more actions to be taken by the user to improve the variability metric of the HRV data.

Clause 19: The non-transitory computer-readable medium of clause 18, wherein the instructions are further executable by the one or more processors to: generate, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

Clause 20: The non-transitory computer-readable medium of any of clauses 18-19, wherein the machine learning model is configured to weight the plurality of HRV values in accordance with a first predictive weight of the one or more predictive weights based at least in part on the variability metric satisfying a threshold value, or wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A system, comprising:
a wearable device configured to measure physiological data from a user via one or more light-emitting components and one or more light-receiving components of the wearable device, the physiological data comprising at least heart rate variability (HRV) data measured continuously from the user throughout a plurality of time intervals of a sleep period that the user is asleep;
a user device communicatively coupled with the wearable device; and
one or more processors communicatively coupled with the wearable device and the user device, the one or more processors configured to:
identify a plurality of HRV values associated with the plurality of time intervals of the sleep period based at least in part on the HRV data collected via the wearable device throughout the sleep period;
determine a variability metric associated with the plurality of HRV values of the sleep period, the variability metric associated with one or more changes in the plurality of HRV values throughout the sleep period;
input, using the one or more processors, the variability metric into a machine learning model, wherein the machine learning model is trained to calculate one or more physiological parameters of the user based at least in part on weighting the plurality of HRV values in accordance with one or more predictive weights that are based at least in part on the variability metric; and
transmit, using the one or more processors, one or more signals to the user device, the one or more signals comprising an instruction for a graphical user interface (GUI) of the user device to display the one or more calculated physiological parameters and a recommendation for one or more actions to be taken by the user to improve the variability metric of the HRV data.

2. The system of claim 1, wherein the one or more processors are further configured to:
generate, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

3. The system of claim 1,
wherein the machine learning model is configured to weight the plurality of HRV values in accordance with a first predictive weight of the one or more predictive weights based at least in part on the variability metric satisfying a threshold value, or
wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

4. The system of claim 3, wherein the second predictive weight is greater than the first predictive weight, and the variability metric satisfies the threshold value based at least in part on the variability metric being greater than the threshold value.

5. The system of claim 1, wherein, to determine the variability metric, the one or more processors are further configured to:
determine a difference between a first percentile of the plurality of HRV values and a second percentile of the plurality of HRV values based at least in part on identifying the plurality of HRV values, wherein the variability metric associated with the plurality of HRV values is based at least in part on the difference.

6. The system of claim 1, wherein, to determine the variability metric, the one or more processors are further configured to:
determine a standard deviation of the plurality of HRV values based at least in part on identifying the plurality of HRV values, wherein the variability metric associated with the plurality of HRV values is based at least in part on the standard deviation.

7. The system of claim 6, wherein, to determine the variability metric, the one or more processors are further configured to:
determine a coefficient of variation for the plurality of HRV values based at least in part on determining the standard deviation, wherein the variability metric associated with the plurality of HRV values is based at least in part on the coefficient of variation.

8. The system of claim 1, wherein the one or more processors are configured to:
identify a stress-inducing event experienced by the user during a time interval preceding the sleep period based at least in part on the variability metric exceeding a threshold value, wherein the one or more signals are configured to cause the user device to display an indication of the stress-inducing event.

9. The system of claim 8, wherein the one or more processors are configured to:
transmit one or more additional signals to the user device, the one or more additional signals configured to cause the user device to display a prompt for the user to confirm the stress-inducing event;
receive, via the user device, a confirmation of the stress-inducing event; and
retrain the machine learning model to identify stress-inducing events for the user based on the HRV data measured from the user based at least in part on inputting the stress-inducing event and the confirmation into the machine learning model.

10. The system of claim 1, wherein the one or more processors are configured to:
train the machine learning model based at least in part on inputting the variability metric into the machine learning model and calculating the one or more physiological parameters of the user.

11. The system of claim 1, wherein the one or more processors are configured to:
train the machine learning model based on a plurality of features within a training physiological dataset associated with a plurality of users, the training physiological dataset comprising a plurality of HRV values associated with the plurality of users.

12. The system of claim 1, wherein the one or more processors are configured to:
determine a plurality of variability metrics associated with a plurality of sleep periods of the user, wherein the variability metric is included within the plurality of variability metrics;
identify a subset of variability metrics that exceed a threshold metric; and
generate an alert provided to the user based at least in part on a quantity of the subset of variability metrics exceeding a threshold quantity, a frequency of the subset of variability metrics exceeding a threshold frequency, or both.

13. The system of claim 1, wherein the wearable device comprises a wearable ring device.

14. A method, comprising:
identifying a plurality of HRV values associated with a plurality of time intervals of a sleep period based at least in part on HRV data collected via a wearable device throughout the sleep period;
determining a variability metric associated with the plurality of HRV values of the sleep period, the variability metric associated with one or more changes in the plurality of HRV values throughout the sleep period;
inputting, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric; and
transmitting, using the one or more processors, one or more signals to a user device, the one or more signals comprising an instruction for a graphical user interface (GUI) of the user device to display the one or more calculated physiological parameters and a recommendation for one or more actions to be taken by the user to improve the variability metric of the HRV data.

15. The method of claim 14, further comprising:
generating, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

16. The method of claim 14,
wherein the machine learning model is configured to weight the plurality of HRV values in accordance with a first predictive weight of the one or more predictive weights based at least in part on the variability metric satisfying a threshold value, or
wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.

17. The method of claim 16, wherein the second predictive weight is greater than the first predictive weight, and wherein the variability metric satisfies the threshold value based at least in part on the variability metric being greater than the threshold value.

18. A non-transitory computer-readable medium storing code, the code comprising instructions executable by one or more processors to:
identify a plurality of HRV values associated with a plurality of time intervals of a sleep period based at least in part on HRV data collected via a wearable device throughout the sleep period;
determine a variability metric associated with the plurality of HRV values of the sleep period, the variability metric associated with one or more changes in the plurality of HRV values throughout the sleep period;
input, using one or more processors, the variability metric into a machine learning model, wherein the machine learning model is trained to calculate one or more physiological parameters of a user based at least in part on weighting the plurality of HRV values in accordance with one or more predictive weights that are based at least in part on the variability metric; and
transmit, using the one or more processors, one or more signals to a user device, the one or more signals comprising an instruction for a graphical user interface (GUI) of the user device to display the one or more calculated physiological parameters and a recommendation for one or more actions to be taken by the user to improve the variability metric of the HRV data.

19. The non-transitory computer-readable medium of claim 18, wherein the instructions are further executable by the one or more processors to:
generate, via the machine learning model, the one or more physiological parameters of the user based at least in part on inputting the variability metric into the machine learning model, wherein the one or more physiological parameters comprises a sleep staging metric, a readiness score, a recovery metric, a stress metric, a pregnancy-related metric, or a combination thereof.

20. The non-transitory computer-readable medium of claim 18,
wherein the machine learning model is configured to weight the plurality of HRV values in accordance with a first predictive weight of the one or more predictive weights based at least in part on the variability metric satisfying a threshold value, or
wherein the machine learning model is configured to weight the plurality of HRV values in accordance with a second predictive weight of the one or more predictive weights based at least in part on the variability metric failing to satisfy the threshold value, wherein the one or more physiological parameters of the user are calculated based at least in part on weighting the plurality of HRV values in accordance with one of the first predictive weight or the second predictive weight.
